(19) United States
(12) Patent Application Publication
(10) Pub. No.: US 2018/0140584 A1
(43) Pub. Date: May 24, 2018

(54) MACROPINOCYTOSIS IN CANCER

(71) Applicant: Memorial Sloan Kettering Cancer Center, New York, NY (US)

(72) Inventors: Craig B. Thompson, New York, NY (US); Wilhelm Palm, New York, NY (US)

(73) Assignees: Memorial Sloan Kettering Cancer Center, New York, NY (US); Memorial Sloan Kettering Cancer Center, New York, NY (US)

(21) Appl. No.: 15/573,446

(22) PCT Filed: May 13, 2016
(86) PCT No.: PCT/US16/32245

§ 371 Date: Nov. 10, 2017

Related U.S. Application Data
(60) Provisional application No. 62/161,219, filed on May 13, 2015.

(51) Int. Cl. A61K 31/436; A61K 31/366; A61K 31/7036; A61K 45/06; A61K 47/64; A61P 35/00; G01N 33/574

(52) U.S. Cl. CPC A61K 31/436; A61K 31/366; A61K 31/7036; A61K 45/06; G01N 2800/52; A61P 35/00; G01N 33/5748; A61K 2300/00; A61K 47/643

(57) ABSTRACT

Disclosed herein are compositions and methods for detecting and treating cancer. In some embodiments, the cancer is characterized by activation of Ras.

### RELATED APPLICATION INFORMATION

The present application claims priority to and benefit of U.S. provisional patent application 62/161,219 (filed May 13, 2015), the entire contents of which are incorporated by reference herein.

### GOVERNMENT SUPPORT

This invention was made with government support under P01 CA104838 awarded by the National Cancer Institute. The United States Government has certain rights in the invention.

### BACKGROUND

To grow and divide, cells require a supply of nutrients that support energy production and macromolecular synthesis. Despite being surrounded by diverse bioenergetics substrates, mammalian cells preferentially metabolize low molecular weight nutrients such as glucose and amino acids. However, proteins are the most abundant organic constituents of body fluids, their combined amino acid content exceeding the amount of monomeric amino acids in human plasma by several orders of magnitude. If accessible to cells, extracellular proteins have the potential to function as alternative nutrients.

Growth factor signaling pathways stimulate cell cycle progression and also promote nutrient uptake and anabolic metabolism. Cancer cells can exploit abnormal growth factor signaling to support dysregulated anabolic metabolism, which may facilitate survival in nutrient depleted microenvironments.

### SUMMARY

The present disclosure teaches, among other things, that the metabolic adaptation of certain cancer cells to their local microenvironment renders them vulnerable to toxin therapy.

The present disclosure further teaches that certain therapeutic modalities designed to treat cancers in fact can have effects that promote proliferation of certain cells. The present disclosure therefore identifies the source of a problem with such therapeutic modalities, and furthermore provides various solutions.

In some embodiments, the present disclosure provides technologies for identifying tumors that may or may not be susceptible to treatment with mTORC1 inhibitors.

In some embodiments, the present disclosure provides technologies for characterizing tumors, determining appropriate therapeutic regimen(s) for tumor treatment, and optionally implementing such regimen(s).

In some embodiments, the present disclosure provides methods comprising steps of administering to a subject suffering from a cancer characterized by oncogenic activation of Ras protein a therapeutic regimen comprising (i) an mTORC inhibition therapy and (ii) a toxin therapy.

In some embodiments, mTORC inhibition therapy is administered prior to the toxin therapy. In some embodiments, the mTORC inhibition therapy comprises an mTORC1 inhibitor.

In some embodiments, the cancer comprises a solid tumor. In some embodiments, the cancer comprises metastatic cells. In some embodiments, the cancer resides in a microenvironment that is desmoplastic and/or hypovascularized. In some embodiments, the cancer resides in a microenvironment low in nutrients or nutrient depleted.

In some embodiments, the cancer is pancreatic cancer.

In some embodiments, the oncogenic activated Ras protein is selected from the group consisting of K-Ras, H-Ras, N-Ras, and combinations thereof. In some embodiments, the Ras protein is K-Ras.

In some embodiments, the mTORC1 inhibitor is selected from the group consisting of rapamycin/sirolimus, everolimus, temsirolimus, umirolimus, zotarolimus, deforolimus, wortmannin, TOP-216, TAFA93, CCI-779, ABT578, SAR543, ascomycin, FK506, AP23573, AP23464, AP23841, KU-0063794, INK-128, EX2044, EX3855, EX7518, AZD-8055, AZD-2014, Palomid 529, Pp-242, OSI-027 and combinations thereof

In some embodiments, the toxin therapy is selected from the group consisting of cyclophosphamide, chlorambucil, cisplatin, busulfan, melphalan, carmustine, streptozotocin, triethylenemelamine, mitomycin C, methotrexate, etoposide, 6-mercaptopurine, 6-thiocguanine, cytarabine, 5-fluorouracil, dacarbazine, actinomycin D, doxorubicin, daunorubicin, bleomycin, mithramycin, vincristine, vinblastine, paclitaxel, pactitaxel derivatives, cytostatic agents, dexamethasone, prednisone, hydroxyurea, asparaginase, leucovorin, amifostine, dactinomycin, mechlorethamine, streptozocin, cyclophosphamide, lomustine, doxorubicin lipo, gemcitabine, daunorubicin lipo, procarbazine, mitomycin, docetaxel, aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil, and combinations thereof

In some embodiments, the toxin is conjugated to a macropinocytosis substrate. In some embodiments, the macropintocytosis substrate is a soluble protein. In some embodiments, the soluble protein is albumin.

In some embodiments, the toxin therapy is administered at a low dose.

In some embodiments, the therapeutic regimen does not comprise a lysosomal inhibition therapy. In some embodiments, the therapeutic regimen does not comprise a Ras inhibition therapy.

In some embodiments, the present disclosure provides methods for identifying a cancer that is likely to respond favorably to treatment with an mTORC1 inhibitor as a monotherapy. In some embodiments, the methods comprise a step of assaying a sample from the cancer for oncogenic activation of Ras, wherein the sample is determined to have low or no oncogenic Ras activity.

In some embodiments, the present disclosure provides methods for identifying a cancer that is likely to not respond favorably to treatment with mTORC1 inhibitor as a monotherapy. In some embodiments, the methods comprise steps of assaying a sample from the cancer for oncogenic activation of Ras, wherein the sample is determined to have oncogenic Ras activity.

In some embodiments, oncogenic activation of Ras comprises constitutively active Ras caused by a genetic mutation. In some embodiments, the genetic mutation comprises a mutated Ras protein. In some embodiments, the genetic mutation results in decreased expression or activity of a Ras suppressor protein.

In some embodiments, oncogenic activation of Ras is detected by allele-specific polymerase chain reaction (PCR), PCR and Sanger dideoxy sequencing, PCR and pyrosequencing, PCR and mass spectrometry (MS), PCR and single base extension, multiplex ligation-dependent probe amplification (MLPA), or fluorescence in situ hybridization (FISH).

The present disclosure also provides compositions for detection of cancer. In some embodiments, compositions for the detection of cancer comprise an imaging agent conjugated to a substrate for macropinocytosis by a cancer cell.

In some embodiments, the compositions further comprise an mTORC1 inhibitor.

In some embodiments, the cancer is detected in vivo in a subject.

In some embodiments, the imaging agent is metallic. In some embodiments, the imaging agent is radiolabeled.

In some embodiments, the substrate for micropinocytosis comprises a soluble protein. In some embodiments, the soluble protein is albumin.

In some embodiments, the detected cancer exhibits oncogenic Ras activity.

In some embodiments, the present disclosure provides methods for detecting cancer in a subject. In some embodiments, the methods comprise steps of: (i) administering to the subject an mTORC1 inhibitor; (ii) administering to the subject an imaging agent capable of eliciting a detectable signal, and (iii) detecting the signal in elicited by the imaging agent.

In some embodiments, the imaging agent is conjugated to a substrate for macropinocytosis by a cancer cell. In some embodiments, the imaging agent is conjugated to a soluble protein. In some embodiments, the soluble protein is albumin.

In some embodiments, the mTORC1 inhibitor is administered prior to the imaging agent. In some embodiments, the mTORC1 inhibitor is administered to the subject at least 1, 3, 5, 12, or 24 hours prior to the imaging agent.

### BRIEF DESCRIPTION OF THE DRAWINGS

The following figures are presented for the purpose of illustration only, and are not intended to be limiting.

FIGS. 1A-C show that physiological levels of extracellular proteins provide nutritional benefits for wild type and K-Ras mutant cells. FIG. 1A shows an exemplary graph of cell numbers of wild type and heterozygous K-Ras^{G12D} MEFs at day 3 of culture in medium±3% albumin lacking different nutrients as indicated [glucose, non-essential amino acids (NEAA), glutamine, essential amino acids (EAA), leucine]. The dashed line indicates starting cell numbers. # below detection limit. * p<0.05, ** p<0.01, *** p<0.001. FIG. 1B shows an exemplary growth curve of wild type and K-Ras^{G12D} MEFs in leucine-free medium±3% albumin. FIG. 1C shows an exemplary growth curve of wild type MEFs expressing myristoylated Akt1 (myr-Akt1) in leucine-free medium±3% albumin. Data are represented as mean±STDEV (n=3).

FIGS. 2A-D show exemplary growth curves. FIG. 2A shows a growth curve of wild type and K-Ras^{G12D} MEFs in EAA-free medium±3% albumin. FIG. 2B shows a growth curve of wild type MEFs expressing control or PTEN shRNA in leucine-free medium±3% albumin. FIGS. 2C and 2D show growth curves of wild type MEFs transfected with empty vector or K-Ras^{G12V} (2C) or empty vector or H-Ras^{G12V} (2D) grown, in leucine-free medium±3% albumin. FIGS. 2E and 2F show growth curves of wild type and Ulk 1/2 double knockout (DKO) MEFs transfected with K-RAS^{G12V} (2E) or H-Ras^{G12V} (2F) in leucine-free medium±3% albumin. Expression of constitutively active Ras variants was induced with 50 ng/ml doxycycline. Data are represented as mean±STDEV (n=3).

FIGS. 3A-E show that macropinocytosis and lysosomal degradation of extracellular proteins supports growth of Ras mutant cells during EAA starvation. FIG. 3A shows an exemplary growth curve of wild type and K-Ras^{G12D} MEFs in amino acid-deficient medium containing EAAs at 5% of the levels in complete medium±3% albumin. FIG. 3B shows an exemplary growth curve of K-Ras^{G12D} MEFs in amino acid-deficient medium containing 5% EAAs supplemented with indicated albumin concentrations. FIG. 3C shows uptake and intracellular degradation of albumin in K-Ras^{G12D} MEFs, assessed by fluorescently labeled BSA and DQ-BSA. Protease inhibitors: 2 μM pepstatin A, 2 μM E-64, 10 μM leupeptin. Scale bars=10 μm. FIG. 3D shows an exemplary growth curve of K-Ras^{G12D} MEFs in leucine-free medium±3% albumin and protease inhibitors as in (3C). FIG. 3E shows an exemplary graph of cell numbers of K-Ras^{G12D} MEFs at day 3 of culture in leucine-free medium±3% albumin and 25 μM EIPA. The dashed line indicates starting cell numbers. *** p<0.001. Data are represented as mean±STDEV (n=3).

FIGS. 4A and 4B show exemplary growth curves. FIG. 4A shows a growth curve of wild-type MEFs in amino acid-deficient medium containing EAAs at 5% of the levels in complete medium, supplemented with indicated albumin concentrations. FIG. 4B shows a growth curve of K-Ras^{G12D} MEFs in leucine-free medium±3% albumin and 2004 chloroquine. Data are represented as mean±STDEV (n=3). FIG. 4C shows an effect of K-Ras^{G12D} mutation on uptake of extracellular macromolecules. Wild type and K-RaS^{G12D} MEFs were serum-starved for 16 h, then incubated with fluorescently labeled dextran, and intracellular dextran was quantified after 30 min uptake. Data are presented as mean±STDEV (n=7 fields of view with >10 cells each). P-values were calculated using a two-tailed unpaired t-test *** p<0.001.

FIGS. 5A-F show that lysosomal degradation of internalized proteins activates the mTORC1 pathway. Figure A shows a comparison of mTORC1 activation in wild type and K-Ras^{G12D} MEFs, analyzed by Western Blotting (WB). MEFs were starved of EAAs for 1 h, then placed in medium containing EAAs or 3% albumin, or in fresh EAA-free medium for 4 h. FIG. 5B shows an exemplary time course of mTORC1 activation in K-Ras^{G12D} MEFs by stimulation with 3% albumin after 1 h EAA starvation, analyzed by WB. FIGS. 5C-F show exemplary effects of inhibiting lysosomal function or macropinocytosis on albumin-dependent mTORC1 activation, analyzed by WB. K-Ras^{G12D} MEFs were starved of EAAs for 1 h, then placed in medium containing EAAs or 3% albumin, or in fresh EAA-free medium for 3 h. At the onset of starvation, bafilomycin A1 (5C), lysosomal protease inhibitors (10 μM pepstatin A, 20 μM E-64) (5D), EIPA (Na+/H+ exchange inhibitor) (5E) or IPA-3 (PAK1 inhibitor) (5F) were added.

FIG. 6 shows that extracellular proteins can activate the mTORC1 pathway. FIG. 6A shows mTORC1 activation upon stimulation with albumin at different concentrations in wild type and K-Ras^{G12D} MEFs, analyzed by Western blotting against mTORC1 -specific phosphorylation of S6 kinase 1. If indicated, 500 nM torin 1 was added at the onset of EAA starvation. FIG. 6B shows a comparison of mTORC1 activation by EAAs and albumin in wild type MEFs transfected with empty vector or H-Ras^{G12V}, analyzed by Western blotting. FIG. 6C shows a comparison of mTORC1 activation by EAAs and albumin in wild type MEFs transfected with control or PTEN shRNA, analyzed by Western blotting. FIG. 6D shows a time course of mTORC1 activation by albumin in wild type MEFs, analyzed by Western blotting. FIG. 6E shows an effect of perturbed lysosomal function on albumin-dependent mTORC1 activation, analyzed by Western blotting. Chloroquine was added at indicated concentrations at the onset of starvation. FIGS. 6F and 6G show exemplary effects of macropinocytosis inhibition on albumin-dependent mTORC1 activation, analyzed by Western blotting; cytochalasin D (actin depolymerization inhibitor) (6F) or Jasplakinolide (actin polymerization inhibitor) (6G) at indicated concentration were added at the onset of starvation. In all experiments, mTORC1 was inactivated by subjecting cells to 1 h EAA starvation. MTORC1 reactivation was then monitored by placing cells in a medium containing EAAs or albumin, or in fresh EAA-free medium for 3-4 h.

FIGS. 7A-C show that lysosomal degradation of internalized proteins induces lysosomal recruitment of mTOR. FIG. 7A shows an exemplary image of lysosomal recruitment of mTOR by extracellular proteins or EAAs, analyzed by immunofluorescence against mTOR and the lysosomal marker LAMP2. K-Ras^{G12D} MEFs were starved of EAAs for 1 h, then placed in medium containing EAAs or 3% albumin, or in fresh EAA-free medium for 2 h. FIG. 7B shows an exemplary image of the consequences of inhibiting lysosomal proteolysis on lysosomal recruitment of mTOR by extracellular proteins in K-Ras^{G12D} MEFs treated and analyzed as in (A). 200 nM bafilomycin A1 was added at the onset of EAA starvation. FIG. 7C shows an exemplary image of the consequences of RagA/B knockdown on lysosomal recruitment of mTOR in K-Ras^{G12D} MEFs treated and analyzed as in (A). Scale bars=10 μm.

FIG. 8 shows that Rag GTPases mediate activation of mTORC1 by extracellularly derived proteins. An exemplary Western blot shows consequences of RagA/B knockdown on albumin-dependent mTORC1 activation. K-Ras^{G12D} MEFs expressing control or RagA/B shRNA were subjected to 1 h EAA starvation, then placed in medium containing EAAs or 3% albumin, or in fresh EAA-free medium for 3 h.

FIGS. 9A-E show that mTORC1 signaling is a negative regulator of extracellular protein-dependent growth. FIG. 9A shows a graph of cell numbers of K-Ras^{G12D} MEFs at day 3 of culture in leucine-containing or leucine-free medium+3% albumin and following inhibitors: MEK1/2 (1 μM PD0325901, 50 μM PD98059), PI3-kinase (25 μM LY294002, 2 μM wortmannin), tyrosine kinases (50 μM genistein), mTOR (50 nM rapamycin, 250 nM torin 1), mTOR/PI3-kinase (0.5 82 M BEZ235, 0.5 μM GDC0980). Dashed lines indicate starting cell numbers. FIG. 9B show an exemplary Western blot of mTOR, PI3-kinase and MAP kinase pathway activity in K-Ras^{G12D} MEFs cultured for 1 day in leucine-free medium+3% albumim. Inhibitors were as in (A). FIG. 9C shows an exemplary growth curve of K-Ras^{G12D} MEFs in leucine-free medium±3% albumin and indicated concentrations of torin 1. FIG. 9D shows an exemplary growth curve of K-Ras^{G12D} MEFs expressing shRNA against Raptor, Rictor or control in leucine-free medium±3% albumin. FIG. 9E shows bright field images of K-Ras^{G12D} MEFs expressing shRNA against Raptor, Rictor or control at day 4 of culture in leucine-free medium±3% albumin. Scale bars=50 μm. Data are represented as mean±STDEV (n=3).

FIGS. 10A-C show that mTOR inhibition promotes albumin-dependent cell proliferation during essential amino acid deprivation. FIG. 10A shows an exemplary growth curve of K-Ras^{G12D} MEFs in leucine-free medium±3% albumin and 25 nM rapamycin or 250 nM torin 1. FIG. 10B shows population doublings of K-Ras^{G12D} MEFs at day 3 of culture in leucine-containing or leucine-free medium+3% albumin and indicated concentrations of torin 1. FIG. 10C shows fold change in cell numbers of the K-Ras mutant tumor cell lines (KRPC, MiaPaCa-2, A549) at day 3 of culture in full medium supplemented with 3% albumin±250 nM torin 1, or at day 4 of culture in leucine-free medium supplemented with 3% albumin±250 nM torin 1. Dashed lines indicate starting cell numbers. P-values were calculated using a two-tailed unpaired t-test. *** p<0.001. FIG. 10D shows a growth curve of K-Ras^{G12D} MEFs in medium lacking isoleucine, lysine or arginine±3% albumin and 250 nM torin 1. FIG. 10E shows an exemplary effect of shRNA-mediated depletion of Raptor or Rictor in K-Ras^{G12D} MEFs on mTORC1 and mTORC2 pathway activity, analyzed by Western blotting. Under experimental conditions, cells have abundant glucose supplied in their medium and are thus unlikely to utilize leucine for lipid as opposed to protein synthesis during anabolic growth. Consistent with this, when cells were labeled with ¹³C-leucine, the ratio of protein to lipid incorporation of label was: control cells 364±69, torin-1 treated cells 203±27, raptor-deficient cells 87±3. Data are represented as mean±STDEV (n=3).

FIGS. 11A-D show that mTORC1 suppresses lysosomal degradation of internalized proteins. FIG. 11A shows an exemplary time course of lysosomal DQ-BSA degradation in K-Ras^{G12D} MEFs in the presence or absence of 250 nM torin 1. FIG. 11B shows quantification of DQ-BSA fluorescence of cells shown in (A). FIG. 11C show lysosomal degradation of DQ-BSA in wild type and K-Ras^{G12D} MEFs after 6 h DQ-BSA uptake in the presence or absence of 250 nM torin 1. FIG. 11D shows quantification of DQ-BSA fluorescence of cells shown in (C). Data are represented as mean±STDEV (n≥5 fields of view with>20 cells each). * p<0.05, *** p<0.001. Scale bars=20 μm.

FIGS. 12A-D show that mTORC1 -regulated catabolism of internalized proteins does not depend on changes in endocytosis or gene expression. FIG. 12A shows an exemplary effect of mTOR inhibition on uptake of extracellular macromolecules. K-Ras^{G12D} MEFs were pre-treated for 1 h with 250 nM torin 1 and then incubated with fluorescently labeled dextran or albumin. Intracellular dextran or albumin was quantified after 30 min uptake. Data are represented as mean±STDEV (n=6 fields of view with>10 cells each). P-values were calculated using a two-tailed unpaired t-test. n.s. not significant. FIG. 12B shows an exemplary effect of shRNA-mediated depletion of Raptor on uptake of extracellular dextran by K-Ras^{G12D} MEFs. Uptake of fluorescently labeled dextran was quantified after 30 min uptake. Data are represented as mean±STDEV (n=8 fields of view with>10 cells each). P-values were calculated using a two-tailed unpaired t-test. n.s. not significant. FIG. 12C shows an exemplary time course of dextran uptake. K-Ras^{G12D} MEFs were pre-treated for 1 h with 250 nM torin 1, then incubated with fluorescently labeled dextran for indicated periods of time. Data are represented as mean±STDEV (n=8 fields of view with>10 cells each). FIG. 12D shows an exemplary effect of shRNA-mediated depletion of Raptor or Rictor on lysosomal degradation of internalized DQ-BSA in K-Ras^{G12D} MEFs. Mean fluorescence intensity per cell was determined after 6 h of DQ-BSA uptake. Data are represented as mean±STDEV (n=7 fields of view with>15 cells each). P-values were calculated using a two-tailed unpaired t-test. *** p<0.001, n.s. not significant. FIG. 12E shows an exemplary effect of chloroquine and lysosomal protease inhibitors on lysosomal degradation of internalized DQ-BSA. K-Ras^{G12D} MEFs were pre-treated for 1 h with 250 nM torin 1 and 20 μM chloroquine or protease inhibitors (2 μM pepstatin A, 2 μM E-64, 10 μM leupeptin). Cells were then incubated with DQ-BSA and indicated inhibitors for 6 h. Scale bars=20 μm. DQ-BSA fluorescence intensity is represented as mean±STDEV (n=7 fields of view with>20 cells each). P-values were calculated using a two-tailed unpaired t-test. *** p<0.001. FIG. 12F shows time dependence of torin 1 treatment on lysosomal DQ-BSA degradation. K-Ras^{G12D} MEFs were pre-treated with 250 nM torin 1 for indicated periods of time, then incubated with DQ-BSA and torin 1. Scale bars=20 μm. Quantification of DQ-BSA fluorescence intensity is represented as mean±STDEV (n=5 fields of view with>30 cells each). FIG. 12G shows the effect of transcription inhibition on torin 1-induced DQ-BSA degradation. K-Ras^{G12D} MEFs were pre-treated with 5 μg/ml actinomycin D or 1 μM triptolide for 30 min. Cells were then incubated with DQ-BSA and indicated inhibitors for 6 h. DQ-BSA fluorescence intensity is represented as mean±STDEV (n=6 fields of view with>30 cells each). P-values were calculated using a two-tailed unpaired t-test. N.s. not significant. FIG. 12H shows quantification of DQ-BSA fluorescence in Ulk1/2 wild type and double knockout (DKO) MEFs expressing K-Ras^{G12V} in the presence (left panel) or absence (right panel) of 250 nM torin 1. Data are represented as mean±STDEV (n=5 fields of view with>20 cells each). FIG. 12I shows a Growth curve of Ulk1/2 DKO MEFs expressing K-Ras^{G12V} in leucine-free medium supplemented with 3% albumin±250 nM torin 1. Expression of constitutively active Ras was induced with 50 ng/ml doxycycline. Data are represented as mean±STDEV (n=3).

FIGS. 13A-G show that mTORC1 signaling has opposing effects on cell proliferation in nutrient-rich and nutrient-depleted conditions. FIGS. 13A and 13B show exemplary graphs of cell numbers of K-Ras^{G12D} MEFs±250 nM torin 1 (13A) and expressing Raptor or control shRNA (13B), at day 3 of culture in medium containing 3% albumin and indicated amounts of EAAs. FIG. 13C shows proliferation of pancreatic tumor cells in control and rapamycin-treated KPC mice, analyzed by immunohistochemistry against Ki-67. Scale bars=400 μm; scale bars in blow-ups=50 μm. FIG. 13D shows an exemplary graph quantifying Ki-67-positive tumor cells in outer and inner tumor regions as shown in (C). FIG. 13E shows an exemplary graph of volume increase of pancreatic tumors in control and rapamycin-treated KPC mice, quantified by 3d high-resolution ultrasound. FIG. 13F shows an exemplary growth curve of Raptor KO MEFs in leucine-containing or leucine-free medium±3% albumin. FIG. 13G shows an exemplary graph of cell numbers of wild type MEFs expressing Raptor or control shRNA at day 3 of culture in medium containing 3% albumin and indicated amounts of EAAs. Data in FIGS. 13A, 13B, 13F, and 13G are represented as mean±STDEV (n=3). Dashed lines indicate starting cell number. Data in FIGS. 13D and 13E are represented as mean±SEM (n=5). * p<0.05. ** p<0.01, *** p<0.001.

FIGS. 14A-D show that mTORC1 inhibition promotes cell proliferation during nutrient deprivation. FIG. 14A shows exemplary proliferation of pancreatic tumor cells in inner, avascular and outer, vascularized tumor regions, analyzed by immunohistochemistry against Ki-67, CD31 and phosphor-S6. Scale bars=50 μm. FIG. 14B shows an exemplary effect of shRNA-mediated depletion of Raptor or Rictor in wild type MEFs on mTORC1 and mTORC2 pathway activity, analyzed by Western blotting. FIG. 14C shows an exemplary growth curve of wild type MEFs expressing Raptor or control shRNA in leucine-free medium±3% albumin. FIG. 14D shows an exemplary growth curve of wild type MEFS in leucine-free medium±3% albumin and 25 nM rapamycin or 250 nM torin 1. FIG. 14E shows the effect of genetic deletion of Raptor or Rictor in inducible KO MEFs on mTORC1 and mTORC2 pathway activity 4 days after induction of Cre, analyzed by Western blotting. FIG. 14F shows an exemplary growth curve of Rictor KO MEFs in leucine-containing or leucine-free medium±albumin. Data are represented as mean±STDEV (n=3).

### DEFINITIONS

Unless otherwise indicated, the terms used herein have the person skilled in the art as commonly understood meaning, in order to facilitate understanding of the present disclosure, some terms will be used herein, the following definitions.

In order for the present invention to be more readily understood, certain terms are first defined below. Additional definitions for the following terms and other terms are set forth throughout the specification.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Activating agent: As used herein, the term “activating agent,” or activator, refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control).

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form.

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog a substance that can be generated from the reference substance by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Antagonist: As used herein, the term “antagonist” refers to an agent that i) inhibits, decreases or reduces the effects of another agent, for example that inactivates a receptor; and/or ii) inhibits, decreases, reduces, or delays one or more biological events, for example, activation of one or more receptors or stimulation of one or more biological pathways. In particular embodiments, an antagonist inhibits activation and/or activity of one or more receptor tyrosine kinases. Antagonists may be or include agents of any chemical class including, for example, small molecules, polypeptides, nucleic acids, carbohydrates, lipids, metals, and/or any other entity that shows the relevant inhibitory activity. An antagonist may be direct (in which case it exerts its influence directly upon the receptor) or indirect (in which case it exerts its influence by other than binding to the receptor; e.g., altering expression or translation of the receptor; altering signal transduction pathways that are directly activated by the receptor, altering expression, translation or activity of an agonist of the receptor).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof

Biologically active: As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Cancer: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Combination therapy: The term “combination therapy”, as used herein, refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

Comparable: The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

Detection entity: The term “detection entity” as used herein refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., 3H, 14C, 18F, 19F, 32P, 35S, 135I, 125I, 123I, 64Cu, 187Re, 111In, 90Y, 99mTc, 177Lu, 89Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Determine: It is appreciated by those of skill in the art that “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Diagnostic information: As used herein, diagnostic information or information for use in diagnosis is any information that is useful in determining whether a patient has a disease or condition and/or in classifying the disease or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely to have a disease or condition (such as cancer), state, staging or characteristic of the disease or condition as manifested in the subject, information related to the nature or classification of a tumor, information related to prognosis and/or information useful in selecting an appropriate treatment. Selection of treatment may include the choice of a particular therapeutic (e.g., chemotherapeutic) agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

Dosage form: As used herein, the terms “dosage form” and “unit dosage form” refer to a physically discrete unit of a therapeutic composition to be administered to a subject. Each unit contains a predetermined quantity of active material (e.g., a therapeutic agent such as an anti-receptor tyrosine kinases antibody). In some embodiments, the predetermined quantity is one that has been correlated with a desired therapeutic effect when administered as a dose in a dosing regimen. Those of ordinary skill in the art appreciate that the total amount of a therapeutic composition or agent administered to a particular subject is determined by one or more attending physicians and may involve administration of multiple dosage forms.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is or has been correlated with a desired therapeutic outcome, when administered across a population of patients.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Inhibition therapy: As used herein, “inhibition therapy” refers to administration of an agent that prevents, reduces, suppresses, blocks, reverses, or otherwise antagonizes an activity or function of a particular target entity. As used herein, “Ras inhibition therapy” refers to administration of an agent that inhibits Ras expression, binding, or activity in a Ras signaling pathway. As used herein, “lysosomal inhibition therapy” refers to administration of an agent that prevents, reduces, suppresses, blocks, reverses, or otherwise antagonizes the activity of lysosomes. In some embodiments, lysosomal inhibition is effected by inhibiting uptake of proteins into lysosomes. In some embodiments, lysosomal inhibition is effected by antagonizing one or more lysosomal enzymes.

Isomer: As is known in the art, many chemical entities (in particular many organic molecules and/or many small molecules) can exist in a variety of structural and/or optical isomeric forms. In some embodiments, as will be clear to those skilled in the art from context, depiction of or reference to a particular compound structure herein is intended to encompass all structural and/or optical isomers thereof. In some embodiments, as will be clear to those skilled in the art from context, depiction of or reference to a particular compound structure herein is intended to encompass only the depicted or referenced isomeric form. In some embodiments, compositions including a chemical entity that can exist in a variety of isomeric forms include a plurality of such forms; in some embodiments such compositions include only a single form. For example, in some embodiments, compositions including a chemical entity that can exist as a variety of optical isomers (e.g., stereoisomers, diastereomers, etc.) include a racemic population of such optical isomers; in some embodiments such compositions include only a single optical isomer and/or include a plurality of optical isomers that together retain optical activity.

Low dose: As used herein, a “low dose” or “low dosage” refers to an amount of an agent or compound that is less than that which is typically administered or prescribed for a given therapeutic indication. In some embodiments, a low dose of a cytotoxic agent is an effective dose in an amount lower than that which is approved by a regulatory agency for the treatment of cancer. In some embodiments, a low dose of a cytotoxic agent refers to a dose that is one or more orders of magnitude lower than a reference dose. In some embodiments, a low dose refers to a dose that is one-half, one-third, one-fourth, one-fifth, or more one-sixth, less than a reference dose. In some embodiments, administration of a low dose of a cytotoxic agent results in a reduction or elimination of undesirable side effects without diminished efficacy.

Marker: A marker, as used herein, refers to an agent whose presence or level is a characteristic of a particular tumor or metastatic disease thereof. For example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

Metabolite: As used herein, “metabolite” refers to any substance produced or used during a physical or chemical process within the body that creates or uses energy, such as: digesting food and nutrients, eliminating waste through urine and feces, breathing, circulating blood, and regulating temperature. The term “metabolic precursors” refers to compounds from which the metabolites are made. The term “metabolic products” refers to any substance that is part of a metabolic pathway (e.g., metabolite, metabolic precursor).

Modulator: The term “modulator” is used to refer to an entity whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Mutant: Mutant: As used herein, the term “mutant” refers to an entity that shows significant structural identity with a reference entity but differs structurally from the reference entity in the presence or level of one or more chemical moieties as compared with the reference entity. In many embodiments, a mutant also differs functionally from its reference entity. In general, whether a particular entity is properly considered to be a “mutant” of a reference entity is based on its degree of structural identity with the reference entity. As will be appreciated by those skilled in the art, any biological or chemical reference entity has certain characteristic structural elements. A mutant, by definition, is a distinct chemical entity that shares one or more such characteristic structural elements. To give but a few examples, a small molecule may have a characteristic core structural element (e.g., a macrocycle core) and/or one or more characteristic pendent moieties so that a mutant of the small molecule is one that shares the core structural element and the characteristic pendent moieties but differs in other pendent moieties and/or in types of bonds present (single vs double, E vs Z, etc) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a mutant polypeptide may differ from a reference polypeptide as a result of one or more differences in amino acid sequence and/or one or more differences in chemical moieties (e.g., carbohydrates, lipids, etc) covalently attached to the polypeptide backbone. In some embodiments, a mutant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a mutant polypeptide does not share at least one characteristic sequence element with a reference polypeptide. In some embodiments, the reference polypeptide has one or more biological activities. In some embodiments, a mutant polypeptide shares one or more of the biological activities of the reference polypeptide. In some embodiments, a mutant polypeptide lacks one or more of the biological activities of the reference polypeptide. In some embodiments, a mutant polypeptide shows a reduced level of one or more biological activities as compared with the reference polypeptide.

Nutrient depleted: As used herein, the phrase “nutrient depleted” refers to a cellular microenvironment in which free levels of one or more essential amino acids are low, or substantially absent, from extracellular space.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Progenitor cell: As used herein, the term “progenitor cell” refers to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate.

Prognostic and predictive information: As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Reference: The term “reference” is often used herein to describe a standard or control agent or value against which an agent or value of interest is compared. In some embodiments, a reference agent is tested and/or a reference value is determined substantially simultaneously with the testing or determination of the agent or value of interest. In some embodiments, a reference agent or value is a historical reference, optionally embodied in a tangible medium. Typically, as would be understood by those skilled in the art, a reference agent or value is determined or characterized under conditions comparable to those utilized to determine or characterize the agent or value of interest.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of

Cancer, National Cancer Institute of the United States, National Cancer Institute of Canada, J. Natl. Cancer Inst., 2000, 92(3):205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Risk: As will be understood from context, a “risk” of a disease, disorder or condition is a degree of likelihood that a particular individual will develop the disease, disorder, or condition. In some embodiments, risk is expressed as a percentage. In some embodiments, risk is from 0, 1, 2, 3, 4, 5, 6, 7, 8, 9, 10 up to 100%. In some embodiments risk is expressed as a risk relative to a risk associated with a reference sample or group of reference samples. In some embodiments, a reference sample or group of reference samples have a known risk of a disease, disorder, or condition. In some embodiments a reference sample or group of reference samples are from individuals comparable to a particular individual. In some embodiments, relative risk is 0, 1, 2, 3, 4, 5, 6, 7, 8, 9, 10 or more.

Sample: As used herein, a sample obtained from a subject may include, but is not limited to, any or all of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification or reverse transcription of mRNA, etc.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic.

Specific: The term “specific”, when used herein with reference to an agent or entity having an activity, is understood by those skilled in the art to mean that the agent or entity discriminates between potential targets or states. For example, an agent is said to bind “specifically” to its target if it binds preferentially with that target in the presence of competing alternative targets. In some embodiments, the agent or entity does not detectably bind to the competing alternative target under conditions of binding to its target. In some embodiments, the agent or entity binds with higher on-rate, lower off-rate, increased affinity, decreased dissociation, and/or increased stability to its target as compared with the competing alternative target(s).

Subject: By “subject” is meant a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. A subject can be a patient, which refers to a human presenting to a medical provider for diagnosis or treatment of a disease. In some embodiments, a subject is an individual to whom therapy is administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition has been diagnosed with and/or exhibits or has exhibited one or more symptoms or characteristics of the disease, disorder, or condition.

Susceptible to: An individual who is “susceptible to” a disease, disorder, or condition is at risk for developing the disease, disorder, or condition. In some embodiments, such an individual is known to have one or more susceptibility factors that are statistically correlated with increased risk of development of the relevant disease, disorder, and/or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from allergy, etc.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. For example, many cancer patients with smaller tumors have no symptoms. It is not intended that the present invention be limited only to cases where the symptoms are eliminated. The present invention specifically contemplates treatment such that one or more symptoms is/are reduced (and the condition of the subject is thereby “improved”), albeit not completely eliminated.

Therapeutic agent: As used herein, the phrase “therapeutic agent” refers to any agent that has a therapeutic effect and/or elicits a desired biological and/or pharmacological effect, when administered to a subject.

Therapeutically effective amount: As used herein, the term “therapeutically effective amount” refers to an amount of a therapeutic protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Toxin therapy: As used herein, the phrase “toxin therapy” refers to the treatment of a cancer with a cytotoxic agent. In some embodiments, a cytotoxic agent includes a small molecule, protein, polypeptide, antibody, virus, or combinations thereof

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Unit dose: The expression “unit dose” as used herein refers to an amount administered as a single dose or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components alternatively or additionally to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs or additional therapies used in combination or coincidental with specific compound(s) employed, and like factors well known in the medical arts.

### DETAILED DESCRIPTION OF CERTAIN EMBODIMENTS

mTORC1 and mTORC1 Inhibition

The kinase mammalian target of rapamycin complex 1 (mTORC1 ) is a central regulator that coordinates cellular nutrient levels with inputs from growth factor signaling to stimulate anabolic metabolism and growth (Ma and Blenis, 2009; Shimobayashi and Hall, 2014; Zoncu et al., 2011b). mTORC1 is composed of mTOR itself, regulatory-associated protein of mTOR (Raptor), mammalian lethal with SEC13 protein 8(MLST8), PRAS40, and DEPTOR. mTORC1 activity strictly depends on sufficient levels of intracellular amino acids, which induce recruitment of mTORC1 to lysosomal membranes (Hara et al., 1998; Sancak et al., 2010). There, mTORC1 can be activated by further inputs from growth factor signaling. Activated mTORC1 phosphorylates multiple targets that concertedly enhance the generation of biomass. For instance, through phosphorylation of S6 kinase (S6K) and 4E binding protein (4E-BP), mTORC1 increases 5′ cap-dependent protein translation (Ma and Blenis, 2009). Conversely, through inhibition of Unc51-like kinase 1/2 (Ulk1/2), mTORC1 suppresses autophagy, thereby preventing degradation of cellular matter (He and Klionsky, 2009; Mizushima, 2010). By these means, mTORC1 promotes cell growth in response to an environment that provides favorable growth signals as well as ample nutrient supply.

The present invention encompasses the finding that mTORC1 suppresses the ability of mammalian cells to utilize extracellular proteins as a source of amino acids to support proliferation. Even in Ras mutant cells with constitutively activated macropinocytosis, mTORC1 impedes degradation of proteins internalized from the environment. Inhibition of mTORC1 elevates lysosomal catabolism of extracellular proteins and promotes proliferation of cells both during deprivation of amino acids in vitro and within poorly vascularized tumor regions in vivo. By preventing nutritional utilization of extracellular proteins, mTORC1 activity thus couples cell growth to the supply of free amino acids.

Amino acids regulate mTORC1 activity by inducing its recruitment to lysosomal membranes, and it has been proposed that amino acids must be present within the lysosomal lumen to exert this effect (Zoncu et al., 2011a). This mechanism indicates that the lysosome plays a central role in cellular amino acid sensing. The yeast vacuole, which corresponds to the lysosome of metazoan cells, functions as an amino acid storage site, and evidence suggests that the mammalian lysosome also contains high levels of amino acids (Zoncu et al., 2011b). Thus, the regulation of mTORC1 at lysosomal membranes could allow cells to gauge the pool of intracellular amino acids. The present disclosure demonstrates that lysosomal degradation of endocytosed proteins regulates the mTORC1 pathway at the same step as amino acids that were imported from the environment in their monomeric form: both nutrients induce Rag-dependent recruitment of mTORC1 to lysosomal membranes, which permits its subsequent activation by growth factor signaling (Kim et al., 2008; Sancak et al., 2008). Similarly, lysosomal catabolism of intracellular proteins that were delivered through autophagy leads to recruitment of mTORC1 to this organelle (Yu et al., 2010). Regulation of mTORC1 activity at the lysosome constitutes a sensitive mechanism that allows cells to monitor amino acids recovered from proteins that were delivered through endocytosis or autophagy. Conversely, being activated at lysosomal membranes, mTORC1 may be well positioned to function as a regulator of cargo delivery to the lysosome by these membrane trafficking pathways. Notably, recent phosphoproteomics screens have identified endosomal trafficking proteins as a major class of uncharacterized mTORC1 substrates (Hsu et al., 2011; Yu et al., 2011).

Inhibitors of mTORC1 can be categorized as first generation inhibitors or second generation inhibitors. First generation inhibitors include, for example, rapamycin and analogs of rapamycin. Rapamycin was one of the first inhibitors of mTORC1 , and binds to cytosolic FKBP12 to act as a scaffold molecule and allowing it to dock on the FBP regulatory region on mTORC1. Rapamycin is not very water soluble and is not very stable, so rapamycin analogs, called rapalogs, were developed to improve solubility and stability. In recent years, mTORC1 inhibitors have been approved for treatments against cancers such as renal cell carcinoma, mantle cell lymphoma and pancreatic cancer.

The second generation of mTORC1 inhibitors were designed to overcome problems with upstream signaling upon the administration of first generation inhibitors to cells. One disadvantage of first generation inhibitors of mTORC1 is the negative feedback loop from phosphorylated S6K, which can inhibit the insulin RTK via phosphorylation. With diminished activity of this negative feedback loop, upstream regulators of mTORC1 become more active. Also, mTORC2, which is resistant to rapamycin, can act upstream of mTORC1 by activating Akt. Thus, signaling upstream of mTORC1 may remains active upon its inhibition via rapamycin and the rapalogs.

Second generation inhibitors are able to bind to the ATP-binding motif on the kinase domain of the mTOR core protein itself and abolish activity of both mTOR complexes. In addition, since the mTOR and the PI3K proteins are both in the same phosphatidylinositol 3-kinase-related kinase (PIKK) family of kinases, some second generation inhibitors have dual inhibition towards the mTOR complexes as well as PI3K, which acts upstream of mTORC1.

Exemplary mTORC1 inhibitors include, but are not limited to, rapamycin/sirolimus, everolimus, temsirolimus, umirolimus, zotarolimus, deforolimus, wortmannin, TOP-216, TAFA93, CCI-779, ABT578, SAR543, ascomycin, FK506, AP23573, AP23464, AP23841, KU-0063794, INK-128, EX2044, EX3855, EX7518, AZD-8055, AZD-2014, Palomid 529, Pp-242, OSI-027 and the like.

The present disclosure sheds light on the puzzling lack of efficacy of mTOR inhibitors as cancer therapeutics. The last several years have witnessed much effort to target the mTORC1 pathway in cancer treatment. These studies have been motivated by the observation that human tumors often display elevated mTORC1 activity, commonly because of mutations in its upstream activators, the PI3-kinase and Ras pathways (Manning and Cantley, 2007; Pylayeva-Gupta et al., 2011; Zoncu et al., 2011b). However, recent clinical trials showed only limited efficacy of rapamycin analogs (rapalogs) in a variety of solid tumors (Fruman and Rommel, 2014; Rodon et al., 2013). The present disclosure reveals an intrinsic weakness of mTOR inhibitors in cancer treatment. By enhancing catabolism of extracellularly derived proteins, mTOR inhibitors increase the range of nutrients accessible to a cell to support survival and sustain growth. This may be particularly important in nutrient-depleted tumor microenvironments or during metastasis, when tumor cells must adapt to novel metabolic niches.

### Macropinocytosis

Macropinocytosis is an evolutionarily conserved, non-selective form of endocytosis that can be triggered by Ras GTPases (Bar-Sagi and Feramisco, 1986; Mercer and Helenius, 2009). Macropinocytosis allows unicellular amoeboid eukaryotes to live on extracellular macromolecules, but whether it functions in nutrient acquisition of metazoan cells is not well understood (Amyere et al., 2002). It was recently shown that by promoting macropinocytosis, oncogenic K-Ras signaling could reduce the dependence of proliferating cancer cells on exogenous glutamine supply (Commisso et al., 2013). This suggests that catabolism of extracellular proteins can provide anaplerotic substrates that allow mammalian cells to sustain mitochondrial bioenergetics and suppress apoptosis.

The present disclosure demonstrates that mTOR inhibition can facilitate lysosomal degradation/catabolism of substances taken up extracellularly by macropinocytosis, particularly in cancers characterized by oncogenic activation of Ras protein. The present disclosure further shows that cancers characterized by oncogenic activation of Ras protein may be selectively vulnerable to treatment with mTORC1 inhibitors incombination with toxins.

### Targeted Cancers

The cells of metazoan organisms are instructed by external cues to engage in nutrient uptake. Growth factor signaling pathways not only stimulate cell cycle progression, but also promote nutrient uptake and initiate anabolic metabolism, thereby ensuring sufficient availability of building blocks for the synthesis of macromolecules to increase cellular mass (Thompson, 2011). This principle is exploited by cancer cells, which rely on constitutively activated growth factor signaling to support the dysregulated anabolic metabolism characteristic of transformed cells (Vander Heiden et al., 2009). Several growth factor-directed signaling pathways enhance cellular uptake of low molecular weight nutrients by increasing expression or surface presentation of metabolite transporters. For instance, both the PI3-kinase/Akt and Ras signaling pathways enhance glucose uptake (Manning and Cantley, 2007; Pylayeva-Gupta et al., 2011; Yun et al., 2009).

The present disclosure relates particularly to cancers characterized by oncogenic activation of Ras, wherein the cancer cells exist in a hypoxic and or nutrient-depleted environment. In some embodiments, the cancer cells exist in a hypoxic and or nutrient-depleted environment due to tumor size. In some embodiments, the cancer cells exist in a hypoxic and or nutrient-depleted environment due to a lack of surrounding vasculature (hypovascularized). In some embodiments, the cancer cells are metastatic cells. In some embodiments, the tumor cells are pancreatic cancer cells. In some embodiments, the cancer cells are characterized by oncogenic activation of Ras, wherein the oncogenic activation of Ras comprises constitutively active Ras caused by a genetic mutation. In some embodiments, Ras is selected from the group consisting of KRas, HRas, NRas, and combinations thereof. In some embodiments, the cancer cells are characterized by oncogenic activation of K-Ras.

### Treating Cancers

The present disclosure relates particularly to the treatment of cancers characterized by oncogenic activation of Ras. In some embodiments, the treatment of a cancer characterized by oncogenic activation of Ras comprises administering to a subject a therapeutic regimen comprising an mTORC inhibition therapy and a toxin therapy. In some embodiments, mTORC inhibition therapy is administered prior to the toxin therapy. In some embodiments, the mTORC inhibition therapy comprises an mTORC1 inhibitor.

In some embodiments, a cancer therapy comprises a toxin that accumulates in the lysosome of a cancer cell characterized by oncogenic activation of Ras but does not poison the lysosome. In some embodiments, a cancer therapy does not inhibit lysosomal activity. In some embodiments, a cancer therapy does not inhibit Ras activation.

In some embodiments, a cancer therapy comprises a toxin therapy, wherein the toxin is selected from the group consisting of cyclophosphamide, chlorambucil, cisplatin, busulfan, melphalan, carmustine, streptozotocin, triethylenemelamine, mitomycin C, methotrexate, etoposide, 6-mercaptopurine, 6-thiocguanine, cytarabine, 5-fluorouracil, dacarbazine, actinomycin D, doxorubicin, daunorubicin, bleomycin, mithramycin, vincristine, vinblastine, paclitaxel, pactitaxel derivatives, cytostatic agents, dexamethasone, prednisone, hydroxyurea, asparaginase, leucovorin, amifostine, dactinomycin, mechlorethamine, streptozocin, cyclophosphamide, lomustine, doxorubicin lipo, gemcitabine, daunorubicin lipo, procarbazine, mitomycin, docetaxel, aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil, and combinations thereof. In some embodiments, the toxin therapy is administered at a low dose.

The present disclosure also relates to determining an appropriate therapeutic regimen for a cancer patient depending on characteristics of the cancer. In some embodiments, a cancer is identified as being likely to respond favorably to treatment with an mTORC1 inhibitor as a monotherapy, for example, a cancer with no or low oncogenic Ras activity. In some embodiments, a cancer is identified as being likely to not respond favorably to treatment with mTORC1 inhibitor as a monotherapy, for example, a cancer with oncogenic Ras activity.

In some embodiments, a cancer with oncogenic Ras activity is identified as susceptible to treatment with an mTORC1 inhibitor and a toxin. In some embodiments, a cancer with oncogenic Ras activity is identified as selectively vulnerable to a low dose of a toxin combined with an mTORC1 inhibitor.

Identifying and/or Characterizing Tumor Cells

The present disclosure relates to identifying cancer cells utilizing macropinocytosis to take up nutrients and/or other substances from extracellular space. The present disclosure also relates to compositions suitable for detecting cancer. In some embodiments, compositions suitable for detecting cancer comprise an imaging agent conjugated to a substrate for macropinocytosis by a cancer cell. In some embodiments, compositions suitable for the present invention further comprise an mTORC1 inhibitor. In some embodiments, the cancer is detected in vivo in a subject. In some embodiments, the imaging agent is metallic. In some embodiments, the imaging agent is radiolabeled.

In some embodiments, a therapeutic regimen comprising an mTORC1 inhibitor and a toxin is administered to a subject to treat a cancer identified by an imaging agent to be utilizing micropinocytosis.

### EXEMPLIFICATION

### Reagents

Antibodies were from: Abcam (ab13524 LAMP2); Cell Signaling (#2215 phospho-S240/244 S6, #2217 S6, #2280 Raptor, #2708 S6K1, #2855 phospho-T37/46 4E-BP1, #2920 Akt, #2983 mTOR, #4060 phospho-S473 Akt, #4377 phospho-T389 S6K1, #4856 phospho-S235/236 S6, #6888 phospho-S757 Ulk1, #9101 phospho-T202/Y204 Erk1/2, #9107 Erk1/2, #9476 Rictor, #9552 PTEN, #11817 phospho-S476 Grb10), Dianova (DIA - - - 310 CD31), Santa Cruz (sc-1026 Grb10), Vector Laboratories (VP-K451 Ki-67). Secondary antibodies were from: Life Technologies (Alexa Fluor 488 anti-rabbit, Alexa Fluor 555 anti-rat), GE Healthcare (HRP-linked anti-rabbit, HRP-linked anti-mouse), Vector Labs (VectaStain ABC Kit). Alexa Fluor 647-BSA, Alexa Fluor 488, 10 kDa Dextran, DQ Green BSA, Hoechst 33342, LysoTracker Red and Prolong Antifade+DAPI were from Life Technologies.

Inhibitors were from: EMD Chemicals (rapamycin), Millipore (jasplakinolide), Pfizer (Rapamune), Selleck (BEZ235, GDC0980), Sigma (bafilomycin A1, chloroquine, cytochalasin D, EIPA, genistein, IPA-3, leupeptin, E-64, pepstatin A, wortmannin), Stemgent (PD0325901), Tocris Bioscience (PD98059, torin 1). Non-essential amino acids (M7145), essential amino acids (M5550) and bovine serum albumin (A1470) were from Sigma.

Cell Lines, Stable Transfection with shRNA and cDNA Constructs

Cre was introduced into SV40 large T-immortalized Lox-Stop-Lox-K-Ras^{G12D} and wild type control MEFs (Tuveson et al., 2004) by infection with adenovirus 5-cytomegalovirus-Cre (Iowa Gene Transfer Core). 200,000 cells in 2 ml culture medium were infected in suspension with 1,000 pfu/cell Adenoviral Cre and plated in 6-well plates. The infection was repeated after 6 h, and 12 h later the medium was replaced with virus-free medium. Successful excision of the transcriptional termination sequence, which leads to K-RasG12D expression, was confirmed by PCR.

For inducible expression, K-Ras^{G12V} and H-Ras^{G12V} cDNAs were subcloned into a modified version of the retroviral vector pTRE-Tight (Clonetech) (Zuber et al., 2011). cDNA expression was induced by addition of 50 ng/ml doxycycline (Sigma) to culture medium. Murine Akt-1 containing a Src myristoylation sequence fused to the N terminus (myr-Akt) in the retroviral vector MIGR1 was described previously (Edinger and Thompson, 2002). Plasmids were co-transfected with retrovirus packaging plasmid into HEK293T cells using Lipofectamin 2000 Transfection Reagent (Life Technologies), fresh media added after 16 h, and viral supernatants collected at 48 h. SV40 large T-immortalized wild type MEFs were infected with viral supernatants and 4 μg/ml polybrene and selected with hygromycin (for pTRE-tight-based constructs) or by fluorescence-assisted sorting of GFP-expressing cells (for MIGR1-based constructs).

shRNA-mediated knockdown was induced by expressing the following lentiviral or retroviral hairpins: RagA TRCN0000077493, TRCN0000077496, RagB TRCN0000102655, TRCN0000102657 (the RNAi Consortium shRNA Library); Raptor Addgene plasmid 213390, Rictor Addgene plasmid 21341 (Thoreen et al., 2009); PTEN (Fellmann et al., 2011). Plasmids were co-transfected with lentivirus or retrovirus packaging plasmids into HEK293T cells using Lipofectamin 2000 Transfection Reagent (Life Technologies), fresh media added after 16 h and viral supernatants collected at 48h. Target cells were infected by addition of viral supernatant and 10 μg/ml polybrene. 24 h after infection, cells were selected with puromycin and experiments conducted 2-3 days after selection.

### Western Blotting

Cells were rinsed with ice-cold PBS and detached from culture plates by incubation with ice-cold trypsin (0.05%). Trypsin was inactivated with ice-cold serum, cells pelleted and rinsed with ice cold PBS. Cells were lysed in ice-cold lysis buffer [50 mM HEPES, pH 7.4, 40 mM NaCl2, 2 mM EDTA, 1 mM Na Orthovandanate, 50 mM NaF, 10 mM Na Pyrophosphate, 10 mM Na Glycerophosphate, 1% Triton X-100, 1× Halt protease and phosphatase inhibitor cocktails (Thermo Scientific)] for 15 min, and soluble lysate fractions isolated by centrifugation at 16,000 g for 10 min. Protein concentrations were determined with the Pierce BCA Protein Assay (Thermo Scientific) and equal amounts of proteins analyzed by SDS gel electrophoresis and Western blotting following standard protocols.

### Fluorescence Microscopy

For immunostainings, cells were rinsed with ice-cold PBS, fixed with 4% formaldehyde in PBS for 15 min and permeabilized with 0.05% Triton X-100 in PBS. After rinsing with PBS, cells were blocked with PBG (0.5% BSA, 0.2% cold water fish gelatin in PBS) for 30 min, incubated with primary antibodies in PBG for 1.5 h, washed two times with PBG+4% normal goat serum and then incubated with secondary antibodies in PBG+4% NGS for 45 min. After three washes with PBS, cells were mounted on microscope slides with Prolong Antifade+DAPI and imaged using a Leica TCS SP5-II.

For live imaging, medium was supplemented with 0.3 mg/ml DQ Green BSA. 1 h prior to imaging, 50 nM LysoTracker Red and 0.2 μg/ml Hoechst were added. Cells were imaged either immediately after addition of DQ-BSA and torin 1, or after 5-6 h incubation using a Zeiss LSM 5 LIVE.

### Immunohistochemistry

Tissues were fixed in 10% neutral buffered formalin for 24 h and transferred to 70% ethanol. Paraffin-embedded tissues were sectioned and processed for immunohistochemistry using standard protocols. Images were acquired using a Perkin Elmer Panoramic Scanner. The proliferative index was determined by quantifying the fraction of Ki-67-positive tumor cells in 6 randomly chosen fields of view of each outer and inner/CD-31-negative tumor regions for each sample.

### ¹⁴ C-Leucine-Labeling of Proteins and Lipids

Cells were cultured in full medium containing 0.5 μCi (0.4%) L-[¹⁴C(U)]-Leucine (Perkin Elmer) for 24 h. Total lipid and protein fractions were isolated essentially according to the Blight-Dyer method (Bligh and Dyer, 1959). Protein pellets were resolubilized in 6 M guanidine hydrochloride, then transferred to scintillation fluid. Organic phase-dissolved lipids were transferred to scintillation vials, and after solvent evaporation, resuspended in scintillation fluid. ¹⁴ 37 C-leucine content of proteins and lipids was quantified by scintillation counting.

### Cell Culture and Nutrient Starvation Experiments

Cell culture experiments were performed at 37° C. and 5% CO2 in DMEM/F12 with 10% dialyzed FBS (molecular weight cut-off 10,000; Gemini Biosystems), 100 U/mL penicillin, 100 μg/mL streptomycin, 5 mM glucose and 2 mM glutamine. For proliferation assays, MEFs were plated in complete medium, 5 h later briefly rinsed and then cultured in starvation medium as indicated. For EAA titration experiments, EAAs were supplemented at indicated percentages of 1× MEM amino acid solution (M5550, Sigma). Cancer cell lines were cultured in complete medium for 1 day prior to starvation. Numbers of adherent cells, which are >95% viable as assessed by Trypan Blue exclusion were determined using a Multisizer 4 Coulter Counter (Beckman). For mTOR activation and localization experiments, cells were rinsed and then incubated in EAA starvation medium (DMEM/F12 lacking all amino acids except glutamine) for 1 h, and subsequently placed in medium supplemented with EAAs (1× MEM amino acid solution), albumin (3%, if not stated otherwise) or fresh EAA-free starvation medium for indicated periods of time.

### Mouse Strains and Rapamycin Treatment

The KPC mouse model (LSL-K-RasG12D/+; LSL-Trp53R172H/+; Pdx-1-Cre) has been described previously (Hingorani et al., 2005). KPC mice develop advanced and metastatic PDA with 100% penetrance, recapitulating the histopathological and clinical features of human PDA. Mice were housed at a 12 h light/12 h dark cycle. All procedures were conducted in accordance with the Institutional Animal Care and Use Committee at CSHL. KPC mice with established tumors of comparable size (7-9 mm in diameter, n=5) as determined by ultrasound (Vevo software, Visualsonics) were treated daily for 8 days by oral gavage with either 5 mg/kg Rapamune (Pfizer) or saline (vehicle control). The last Rapamune administration was given 4 h prior to the endpoint. Tumor volumes were measured from 3d ultrasound images taken on day 4 and 7 of treatment.

### Statistical Analysis

P-values were calculated using a two-tailed unpaired t-test for proliferation and fluorescence microscopy experiments of cultured cells and using the Mann-Whitney nonparametric t-test for analysis of murine tumors.

Ras-Directed Macropinocytosis of Extracellular Proteins Can Sustain Cell Viability and Proliferation during Essential Amino Acid Starvation

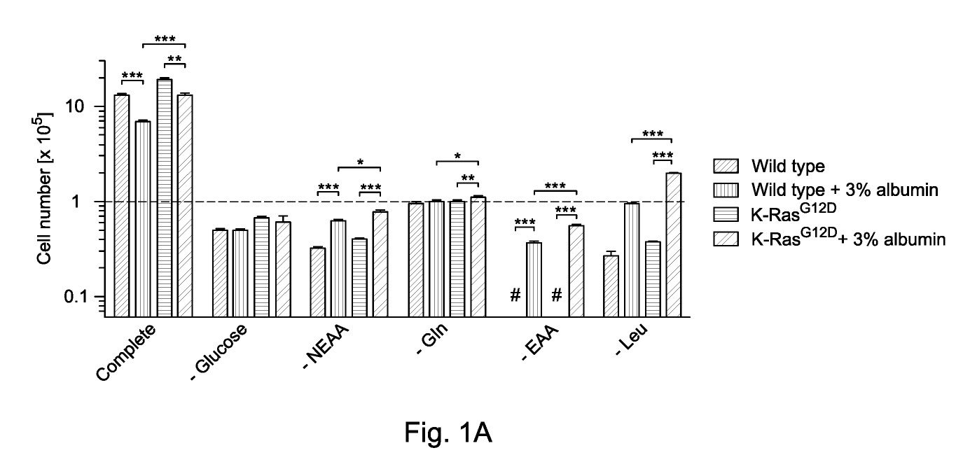

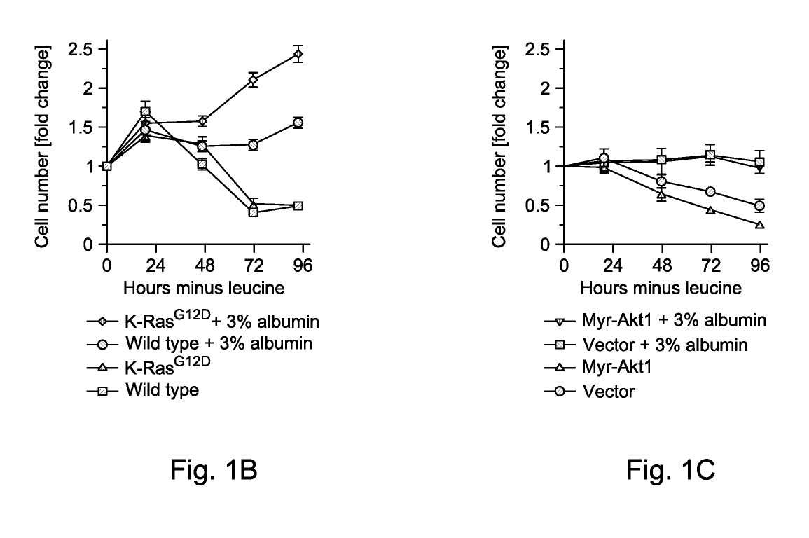

To investigate whether activation of K-Ras signaling altered a cell's ability to metabolize extracellular proteins, we compared immortalized mouse embryonic fibroblasts (MEFs) harboring wild type K-Ras or a constitutively active K-Ras^{G12D} allele. Albumin is the major protein in plasma and interstitial fluids, its levels ranging from 2-5%. To mimic the protein-rich composition of extracellular fluids, culture medium was therefore supplemented with 10% dialyzed fetal bovine serum±3% albumin. When placed in glucose-free medium, both wild type and K-Ras^{G12D} MEFs ceased to proliferate, regardless of the presence of 3% albumin (FIG. 1A). When cells were subjected to non-essential amino acid (NEAA) starvation, albumin supplementation modestly improved viability. When cells were placed in medium lacking a single NEAA, glutamine, albumin supplementation allowed K-Ras^{G12D} MEFs to increase in cell numbers, albeit to a minor extent.

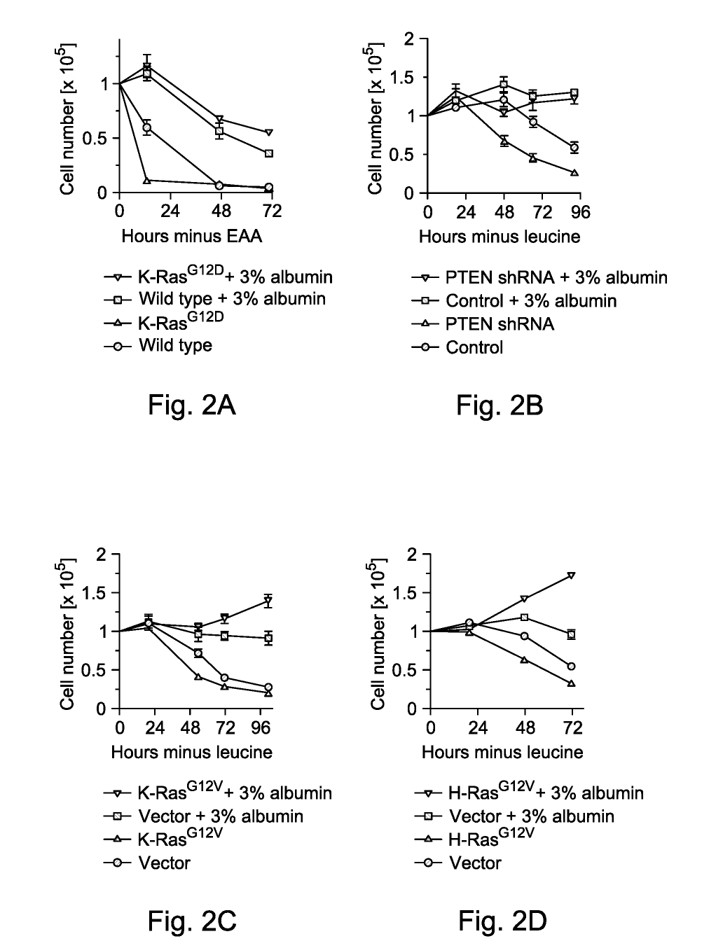

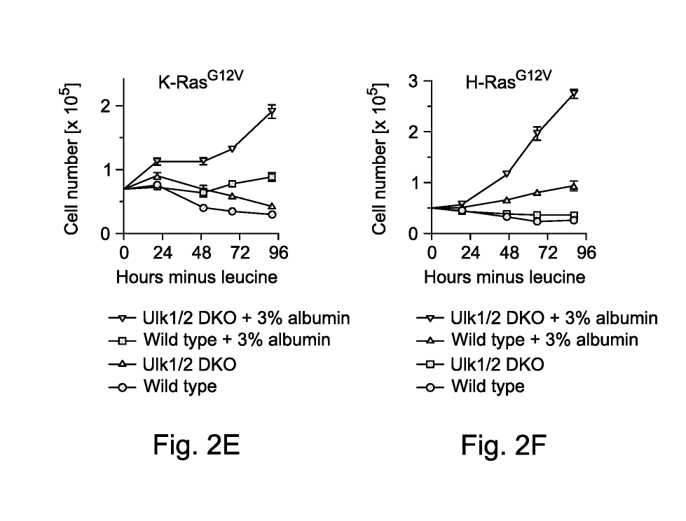

In contrast to the above results, addition of 3% albumin caused a striking rescue of cell survival in medium lacking all essential amino acids (EAAs) (FIG. 1A, FIG. 2A). When starved for a single EAA, leucine, which is the most abundant amino acid in both albumin and the mammalian proteome, viability was completely restored by albumin supplementation (FIG. 1A). K-Ras^{G12D} MEFs were able to sustain proliferation in leucine-free medium containing 3% albumin, with cell numbers increasing over several consecutive days (FIG. 1B). Because growing cells must acquire leucine from exogenous sources to support protein synthesis, these results suggest that K-Ras^{G12D} MEFs could derive leucine from extracellular proteins. However, albumin supplementation of amino acid-deficient medium could sustain only limited cell proliferation as compared to amino acid-replete medium (FIG. 1A).

Besides Ras, PI3-kinase/Akt signaling is a second key pathway that instructs cells to engage in nutrient uptake (Manning and Cantley, 2007). However, expressing myristoylated Akt 1 or PTEN shRNA did not improve the response of cells to albumin supplementation of leucine-free medium (FIG. 1C, FIG. 2B). In contrast, inducing the expression of constitutively active K-Ras^{G12V} or H-Ras^{G12V} supported proliferation under these conditions (FIG. 2C, D), consistent with what we observed in MEFs harboring an endogenous K-Ras^{G12D} allele. The capacity of constitutively activated Ras signaling to sustain moderate levels of proliferation in medium that is protein-rich but amino acid-deficient is consistent with the shared ability of Ras GTPases to enhance macropinocytosis.

One mechanism by which oncogenic Ras signaling has been proposed to sustain cell viability during starvation is increased autophagy to recover nutrients from catabolism of intracellular macromolecules (Pylayeva-Gupta et al., 2011). To examine the role of autophagy in cell growth that utilizes extracellular proteins as a nutrient source, requirements for the autophagy initiator kinases Ulk1/2 were determined. Wild type and Ulk1/2 double knockout (DKO) MEFs expressing K-Ras^{G12V}or H-Ras^{G12V} could sustain proliferation in leucine-free medium when 3% albumin was added as an exogenous amino acid source (FIG. 2E, F). Loss of Ulk1/2 did not impair extracellular protein-dependent cell growth. Instead, Ulk1/2 DKO MEFs proliferated at significantly higher rates than wild type controls. Thus, not only are the autophagy initiator kinases Ulk1/2 dispensable for the utilization of extracellular proteins as nutrients, but loss of Ulk1/2-dependent autophagy actually improves the ability of Ras mutant cells to proliferate under these nutritional conditions.

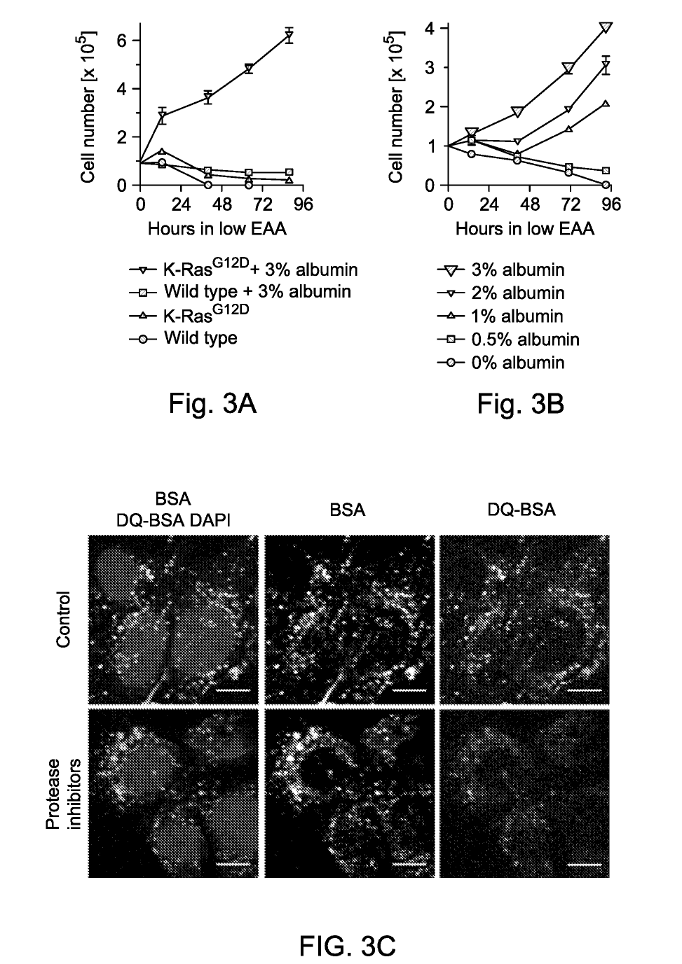

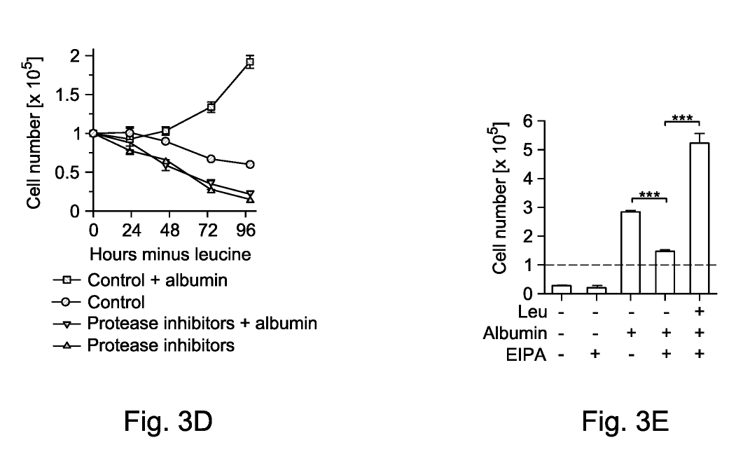

It was next evaluated whether albumin supported cell proliferation in medium containing reduced amounts of all EAAs. When EAAs were supplied at 5% of the levels present in complete medium, wild type and K-Ras^{G12D} MEFs ceased to proliferate and lost viability over time (FIG. 3A). Addition of 3% albumin improved survival of wild type MEFs, but did not support their proliferation. In contrast, albumin supplementation caused a significant increase in proliferation of K-Ras^{G12D} MEFs.

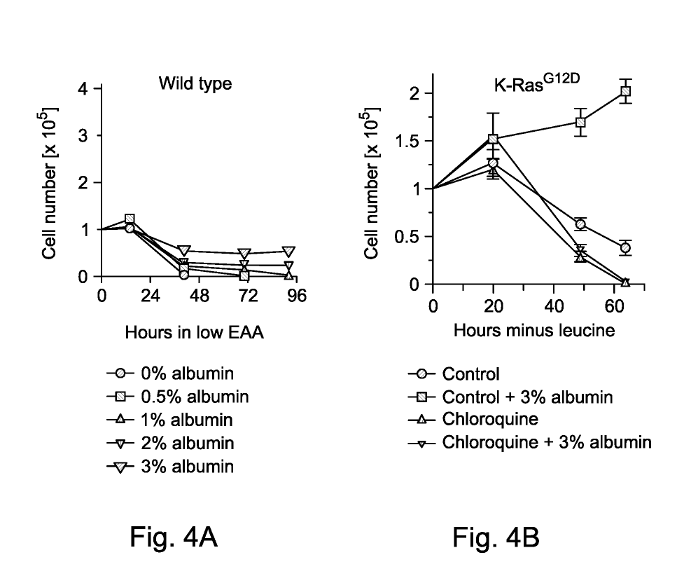

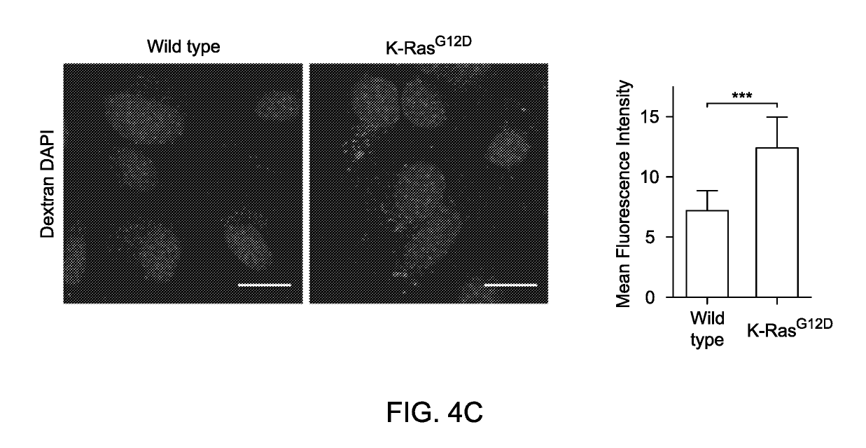

As the proteins provided in 10% dialyzed serum did not suffice to promote cell proliferation during EAA starvation, the concentration of albumin required for proliferation under these conditions was determined. Wild type MEFs cultured in low-EAA medium displayed improved viability in response to increasing albumin levels (1-3%) (FIG. 4A). K-Ras^{G12D} MEFs could sustain proliferation when at least 1% albumin was added, and their proliferation rate increased progressively as the albumin concentration was raised to 3% (FIG. 3B). Thus, albumin must be supplied at physiological levels to support cell survival and growth in EAA-deficient medium. This may explain why the protein-poor media formulations commonly used in cell culture do not suffice for MEFs to proliferate during EAA starvation.

To characterize how cells derive leucine from extracellular proteins, cellular albumin uptake was investigated using constitutively fluorescent albumin and intracellular albumin proteolysis using a self-quenched albumin that emits fluorescence upon degradation (DQ-BSA) (Reis et al., 1998). After 1.5 h label uptake, K-Ras^{G12D} MEFs displayed multiple intracellular structures marked by both albumin and DQ-BSA (FIG. 3C). Adding lysosomal protease inhibitors did not perturb albumin internalization, but caused a strong decrease in DQ-BSA fluorescence. It was then determined whether lysosomal albumin degradation was required to support growth during leucine starvation. Indeed, lysosomal protease inhibitors or the lysosomal acidification inhibitor chloroquine suppressed proliferation of K-Ras^{G12D} MEFs in leucine-free medium containing 3% albumin (FIG. 3D, 4B). Ras GTPases can promote cellular uptake of macromolecules through macropinocytosis (Bar-Sagi and Feramisco, 1986). Consistently, K-Ras^{G12D} MEFs displayed higher macropinocytic activity than wild type controls (FIG. 4C). To investigate whether this endocytic pathway contributed to albumin-dependent growth, cells were treated with the Na⁺/H⁺ exchange inhibitor EIPA, which blocks macropinocytosis but not other endocytic pathways (West et al., 1989). Cell proliferation in leucine-free medium+3% albumin was strongly decreased by EIPA treatment, but restored when free leucine was added to culture medium. (FIG. 3E). Together, these data show that macropinocytic uptake and lysosomal degradation of extracellular proteins can provide nutritional benefits during EAA starvation.

Catabolism of Extracellular Proteins Induces Lysosomal Recruitment and Activation of mTORC1

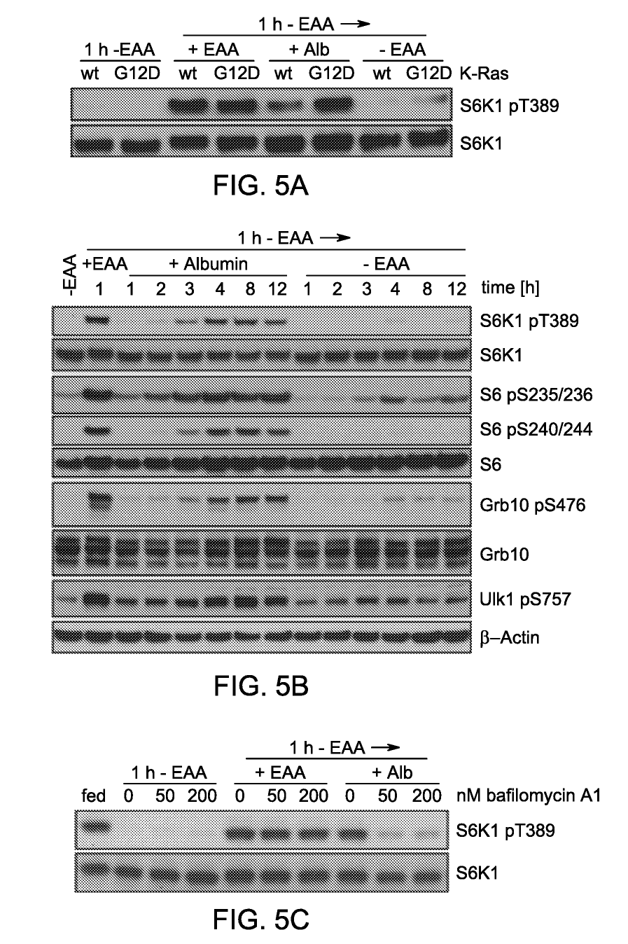

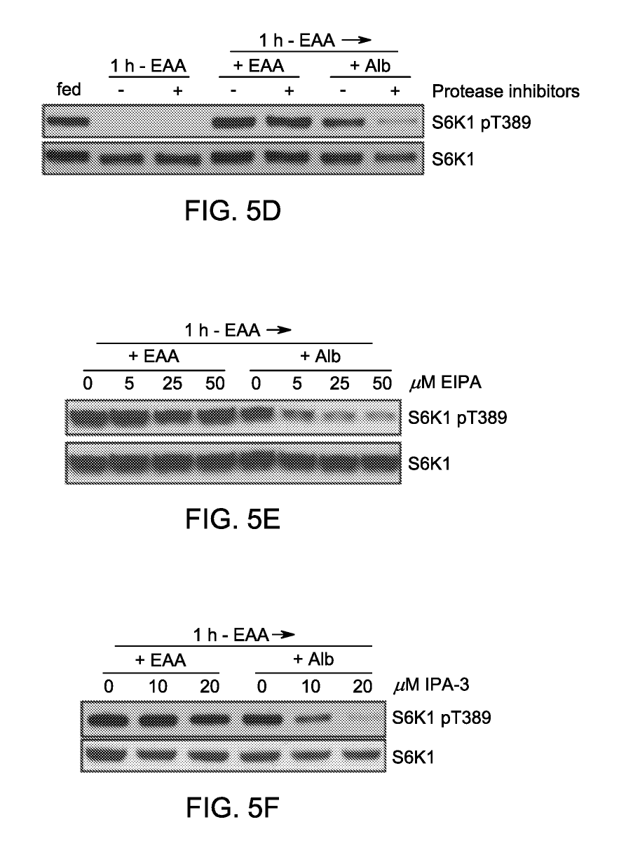

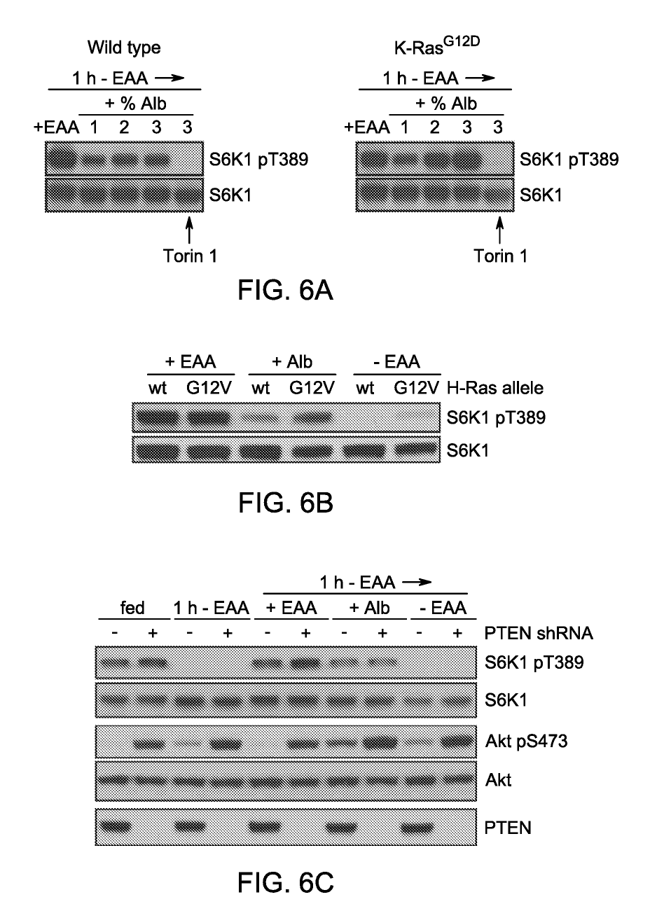

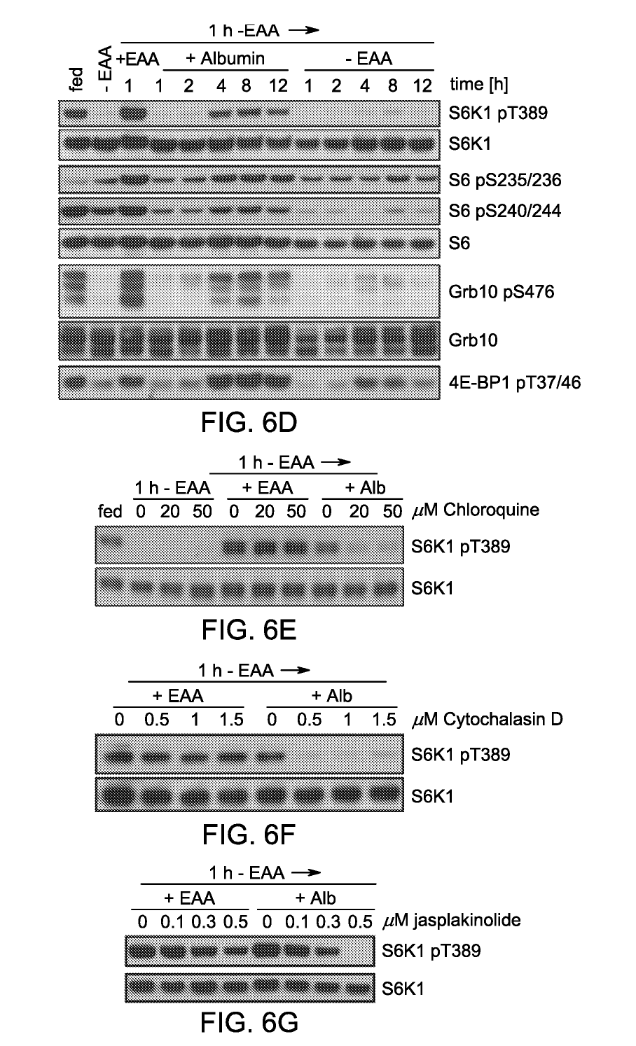

Cells can sense EAAs through the mTORC1 pathway, which integrates amino acid levels with inputs from growth factor signaling to promote cell growth (Ma and Blenis, 2009; Shimobayashi and Hall, 2014). When cells are placed in EAA-free medium, the ability of mTORC1 to phosphorylate downstream targets is repressed. To test if catabolism of extracellularly provided proteins could restore mTORC1 activity in the absence of EAAs, mTORC1 was inactivated by subjecting MEFs to 1 h EAA starvation. Fresh medium containing EAAs or different concentrations of albumin was then added and mTORC1 re-activation assessed by Western blotting against phosphorylated S6K1. Supplementing EAA-free medium with 1-3% albumin caused a concentration-dependent increase in S6K1 phosphorylation in both wild type and K-Ras^{G12D} MEFs, which was completely suppressed by the mTOR inhibitor torin 1 (FIG. 6A). Thus, physiological levels of albumin can activate the mTORC1 pathway. Although wild type and K-Ras^{G12D} MEFs exhibited comparable mTORC1 activation in response to EAAs, K-Ras^{G12D} MEFs displayed higher mTORC1 activity upon addition of 3% albumin (FIG. 5A). Similarly, H-Ras^{G12V} expression increased mTORC1 activity in response to albumin (FIG. 6B). Conversely, elevating PI3Kinase/Akt signaling through sh-RNA mediated depletion of PTEN increased mTORC1 activation by EAAs but not albumin (FIG. 6C). These data suggest that Ras signaling enhances mTORC1 activation in response to albumin stimulation through the amino acid sensing branch rather than the growth factor-regulated branch of the pathway.

Extracellularly provided EAAs are rapidly taken up by cells through transmembrane transporters and activate mTORC1 within minutes (Nicklin et al., 2009). To examine the kinetics of mTORC1 pathway activation by extracellular proteins, mTORC1 re-activation in response to albumin stimulation was followed over time. Re-addition of EAAs caused rapid phosphorylation of mTORC1 targets such as S6K1, Grb10 and Ulk1 as well as the S6K target ribosomal protein S6 (FIG. 3B, 6D) (Kang et al., 2013; Nicklin et al., 2009). In contrast, in response to albumin, mTORC1 -dependent phosphorylation of these proteins resumed only after 2 h and reached maximal levels after 4 h. Thus, extracellular proteins activate the mTORC1 pathway more slowly than EAAs, suggesting that distinct cellular processes are involved in mTORC1 activation by these nutrients.

It was then determined whether macropinocytosis and lysosomal proteolysis were required for mTORC1 activation by extracellular proteins. The lysosomal acidification inhibitors bafilomycin A1 and chloroquine did not perturb mTORC1 activation by EAAs (FIG. 5C, 6E). In contrast, even low concentrations of either inhibitor strongly suppressed albumin-dependent mTORC1 activation. Similarly, protease inhibitors blocked mTORC1 activation in response to stimulation with albumin (FIG. 5D). To establish the relevance of macropinocytosis, the effects of pharmacological inhibitors of macropinocytosis including the Na⁺/H⁺ exchange inhibitor EIPA, the Pak1 inhibitor IPA-3 and the actin inhibitors jasplakinolide and cytochalasin D (Mercer and Helenius, 2009) were investigated. All these inhibitors strongly reduced mTORC1 activation by addition of albumin but not by re-addition of EAAs (FIG. 5E, F; 6F, G).

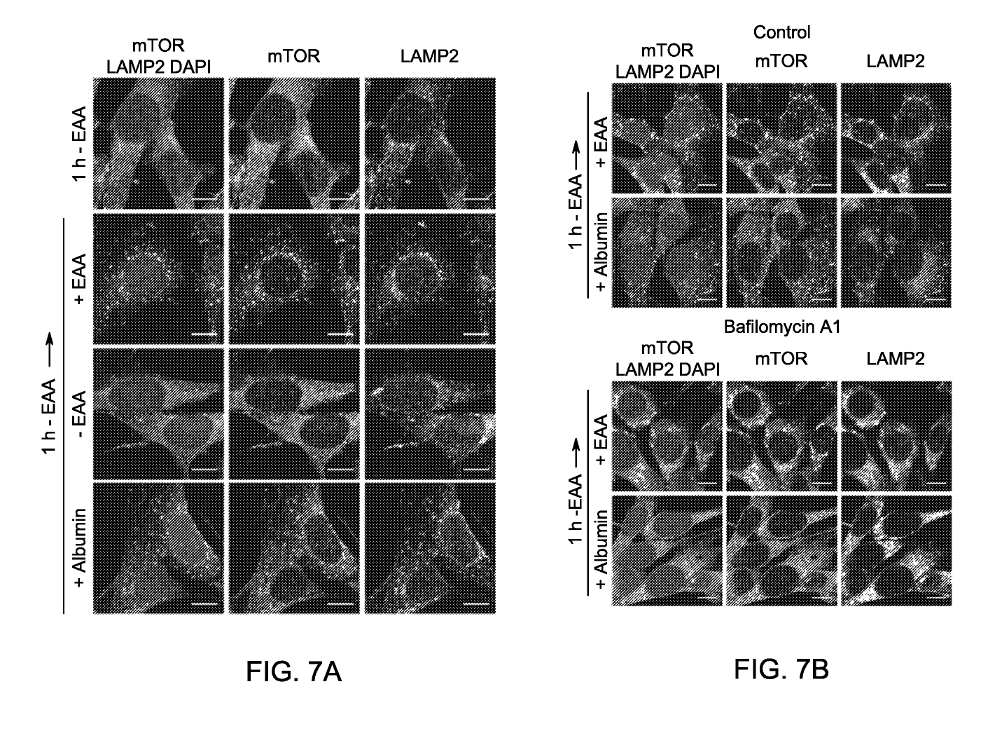

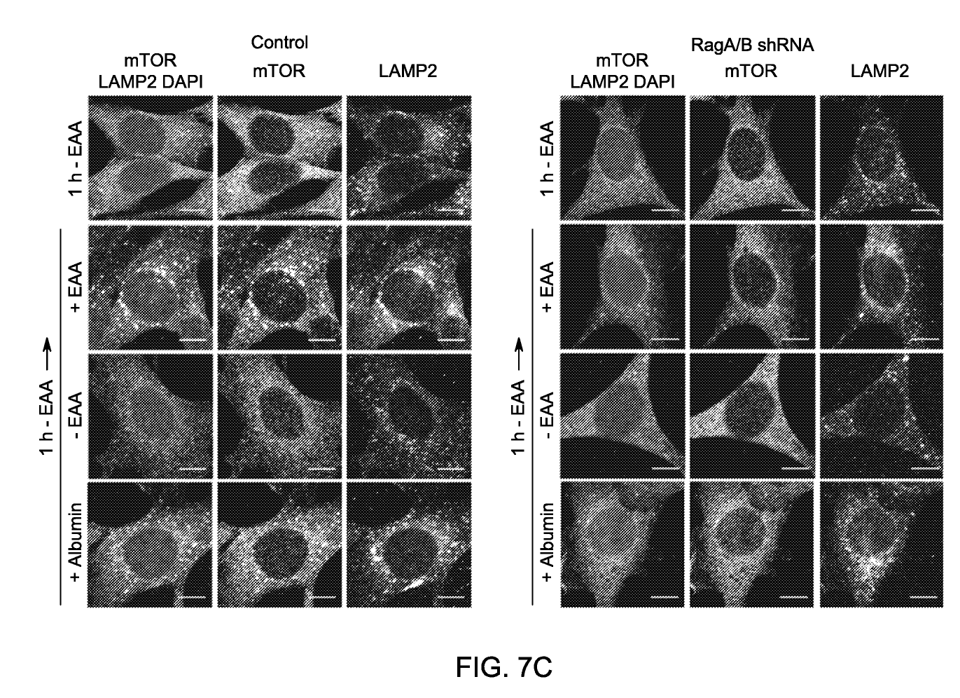

Amino acids signal to mTORC1 by inducing its translocation to lysosomal membranes, where it can be activated by further inputs from growth factor signaling (Sancak et al., 2010). To determine whether extracellular proteins similarly regulated mTORC1 by inducing its recruitment to lysosomes, K-RasG¹²D MEFs were subjected to 1 h EAA starvation, re-fed with EAAs or 3% albumin, and the subcellular localization of mTOR kinase was monitored by immunofluorescence. mTOR was distributed throughout the cytoplasm in EAA-starved cells. Upon albumin addition, mTOR localized to punctate structures that were marked by the lysosomal membrane protein LAMP2 similar to the pattern observed upon re-addition of free EAAs (FIG. 7A). Inhibiting lysosomal proteolysis with bafilomycin A1 completely blocked movement of mTOR to lysosomal membranes in response to albumin but not to EAAs (FIG. 7B).

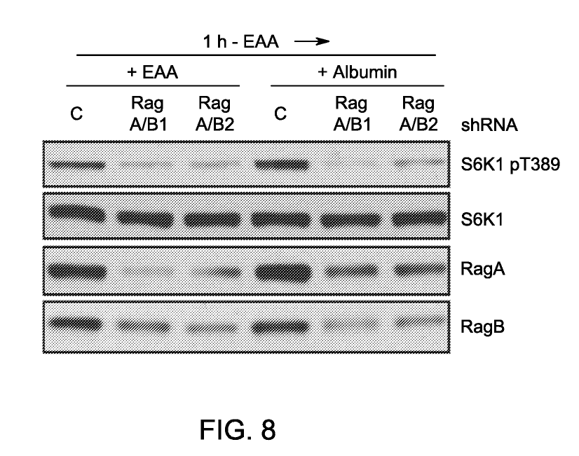

EAAs induce movement of mTORC1 to lysosomal membranes through a mechanism that requires the lysosome-associated Rag GTPases (Kim et al., 2008; Sancak et al., 2008). In contrast, glutamine can activate mTORC1 in a Rag-independent mechanism (Jewell et al., 2015). To investigate whether mTORC1 activation by lysosomal proteolysis of albumin depended on Rag GTPases, the consequences of shRNA-mediated knockdown of RagA and RagB was determined. Depletion of RagA/B resulted in diffuse localization of mTOR throughout the cytosol, regardless whether medium contained albumin or EAAs (FIG. 7C). Consistently, neither albumin nor EAAs could induce mTORC1 -dependent phosphorylation of S6K1 in RagA/B knockdown cells (FIG. 8). Thus, both extracellular proteins and EAAs activate mTORC1 through a Rag-dependent mechanism.

mTORC1 Suppresses Cell Growth that Relies on Extracellular Proteins as Nutrients

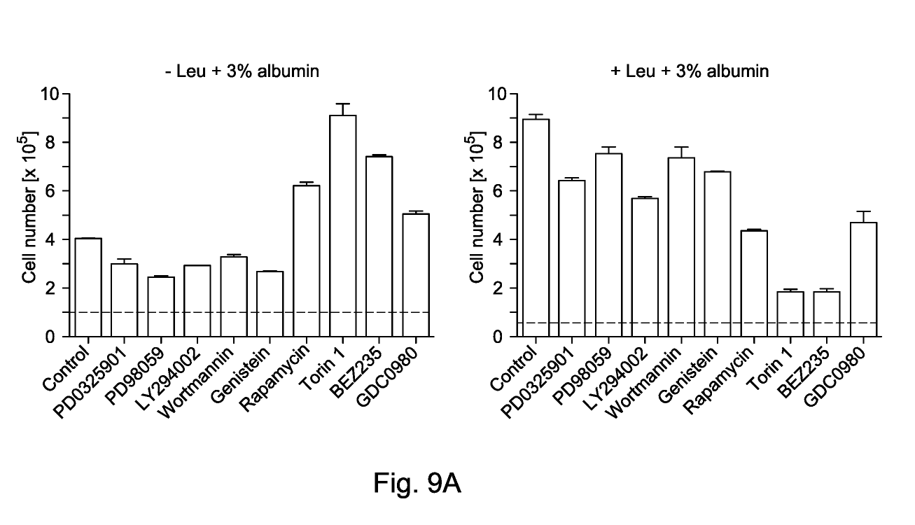

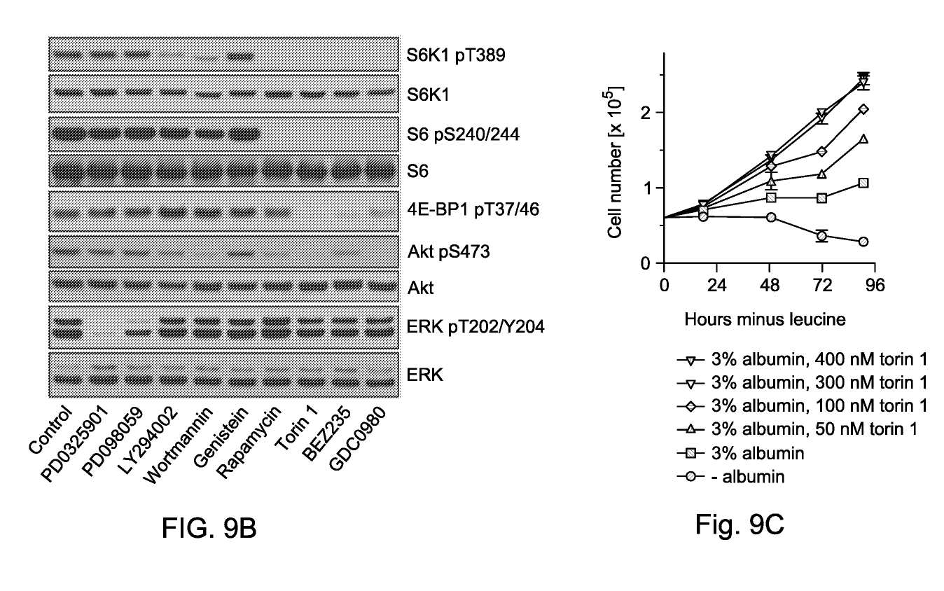

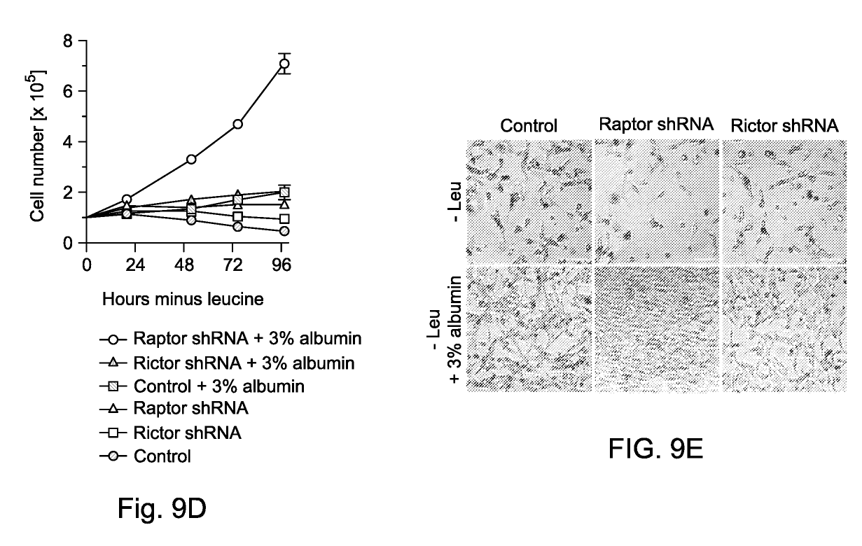

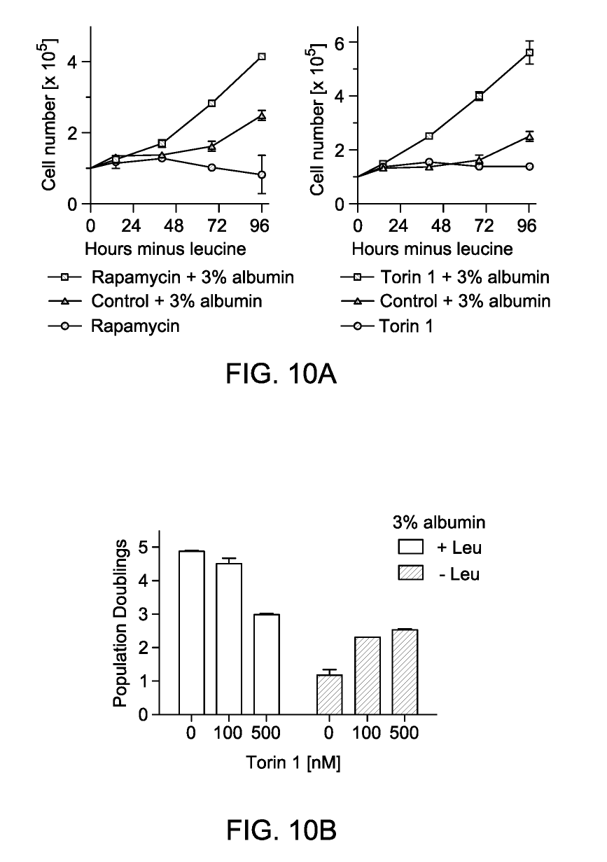

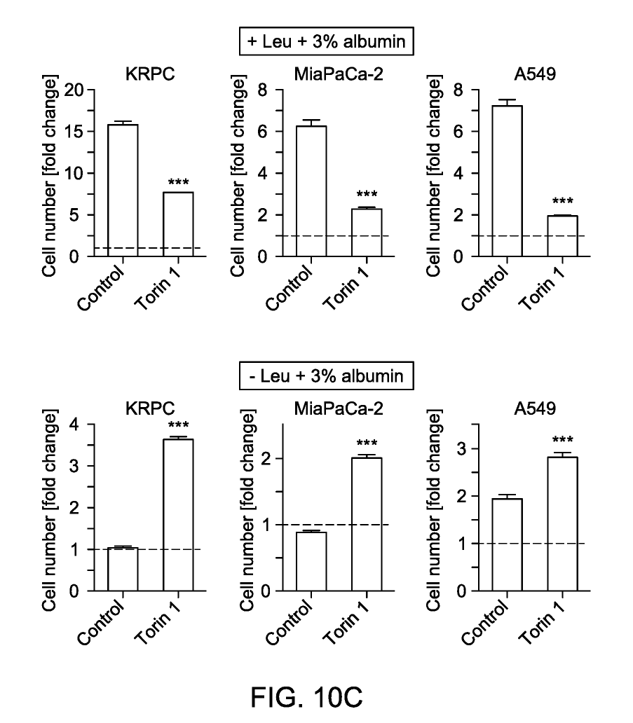

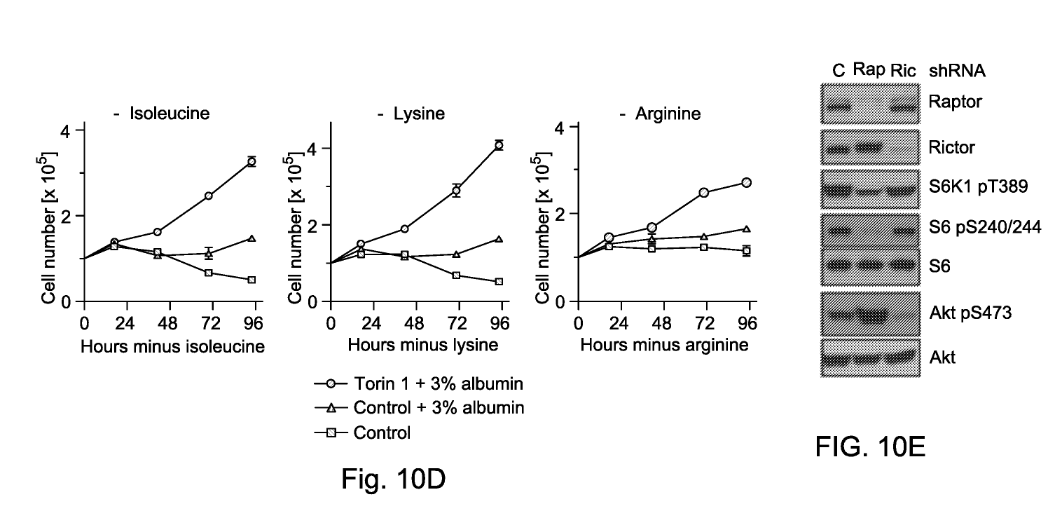

To examine the signaling pathways downstream of Ras that contribute to a cell's ability to utilize extracellular proteins as an amino acid source, the effects of inhibitors against MEK1/2, PI3-kinase, tyrosine kinases and mTOR inhibitors on proliferation of K-Ras^{G12D} MEFs in leucine-free medium plus 3% albumin were determined. Surprisingly, mTOR inhibition did not retard cell proliferation in leucine-free medium supplemented with albumin. Rather, each of the mTOR inhibitors, including mTOR/PI3-kinase dual inhibitors, enhanced proliferation under these conditions (FIG. 9A, left panel, FIG. 10A), although they effectively blocked mTOR signaling activity (FIG. 9B). In contrast, inhibition of MAP kinase, PI3-kinase or tyrosine kinase signaling modestly decreased cell proliferation in leucine-free medium (FIG. 9A, left panel). The effects of these kinase inhibitors were different in leucine-containing medium, where mTOR inhibition was particularly effective as suppressing cell proliferation (FIG. 9A, right panel). Concentration-dependent effects of mTOR inhibitors on cell growth were next examined. Raising concentrations of torin 1 from 50 to 300 nM progressively improved cell proliferation in leucine-free medium +3% albumin, suggesting that proliferation is inversely correlated with mTOR signaling activity when cells use albumin as a source of leucine (FIG. 9C) leucine. In contrast, cell proliferation was significantly higher when free leucine was provided extracellularly, but decreased with increasing doses of torin 1 (FIG. 10B). Similarly, torin 1 treatment induced several carcinoma cell lines harboring activating Ras mutations to proliferate in leucine-free medium +3% albumin, while it strongly decreased their proliferation in leucine-containing medium (FIG. 10C). As albumin provides a mixture of all proteinogenic amino acids, it was also examined whether it could support survival/growth of cells in medium lacking other single EAAs (isoleucine, lysine, or arginine). Indeed, physiological levels of albumin rescued cell viability, and inhibition of mTOR signaling by torin 1 induced cells to robustly proliferate in medium lacking these EAAs (FIG. 10D).

mTOR kinase is present in two distinct complexes: mTORC1, which regulates growth in response to nutrients and growth factor signaling, and mTORC2, which is a component of the PI-3kinase signaling pathway (Shimobayashi and Hall, 2014). To dissect their role in regulating albumin-dependent growth, the essential mTORC1 component, Raptor, or the essential mTORC2 component, Rictor, was depleted in K-Ras^{G12D} MEFs through shRNA-mediated knockdown (FIG. 10E). Rictor knockdown did not enhance cell proliferation during leucine deprivation, regardless of the presence of 3% albumin (FIG. 9D, E). In contrast, Raptor knockdown caused a dramatic increase in proliferation of K-Ras^{G12D} MEFs in leucine-free medium supplemented with albumin, with cells undergoing almost one population doubling per day. Thus, mTORC1 is a negative regulator of cell growth that relies on extracellular proteins as an amino acid source.

mTORC1 Inhibition Enhances Lysosomal Degradation of Internalized Albumin

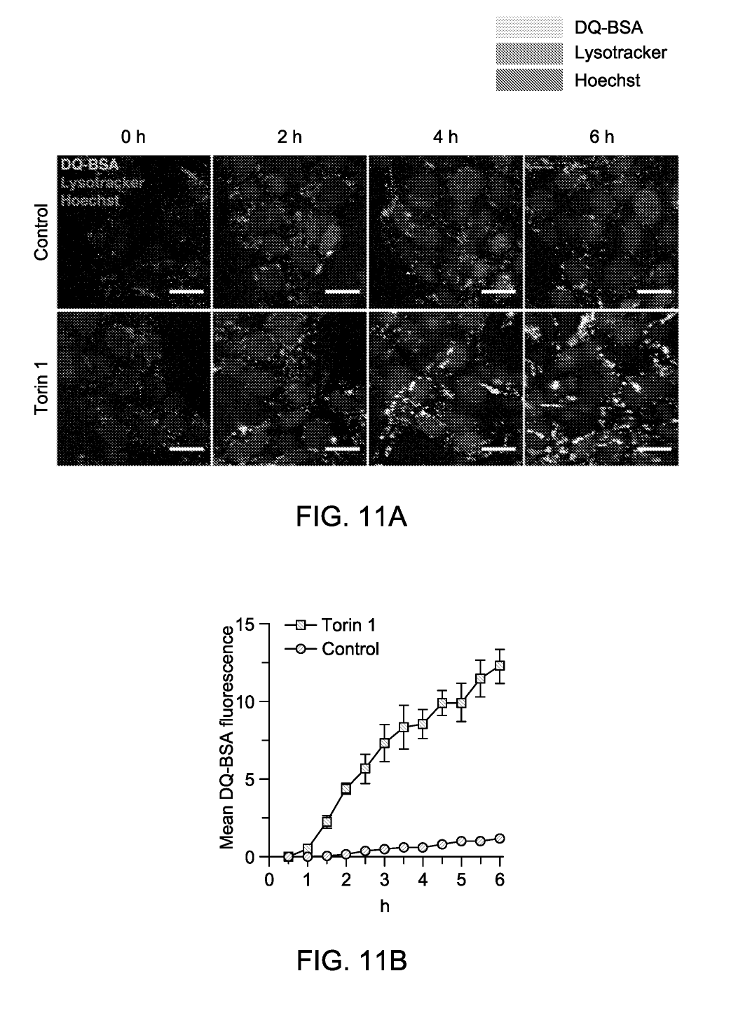

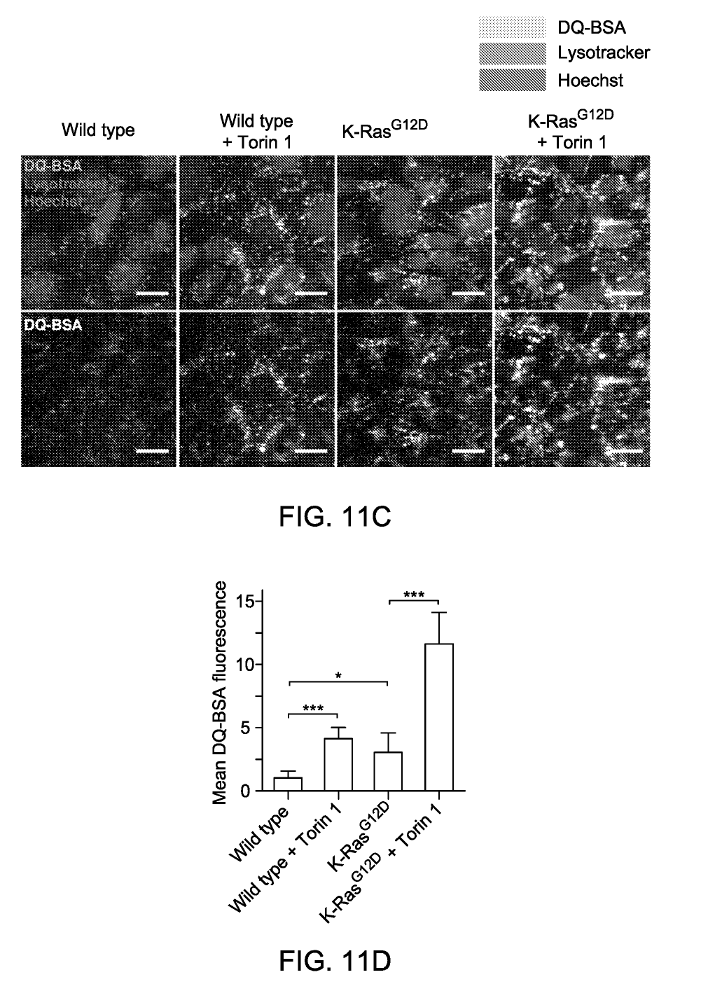

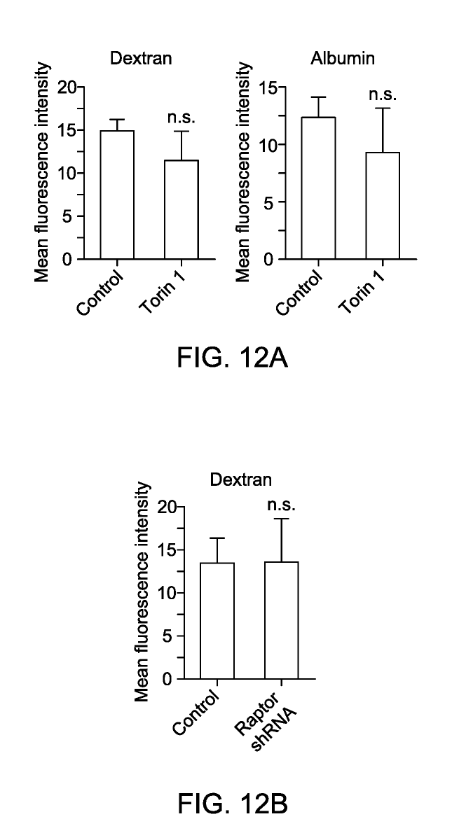

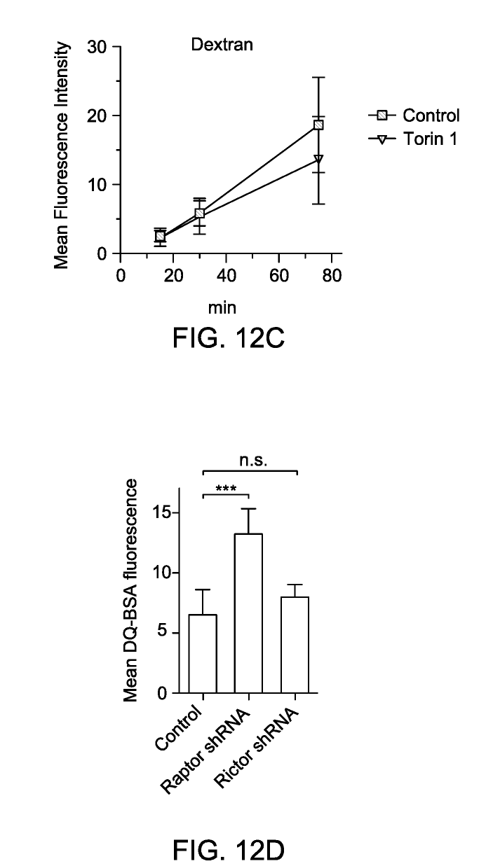

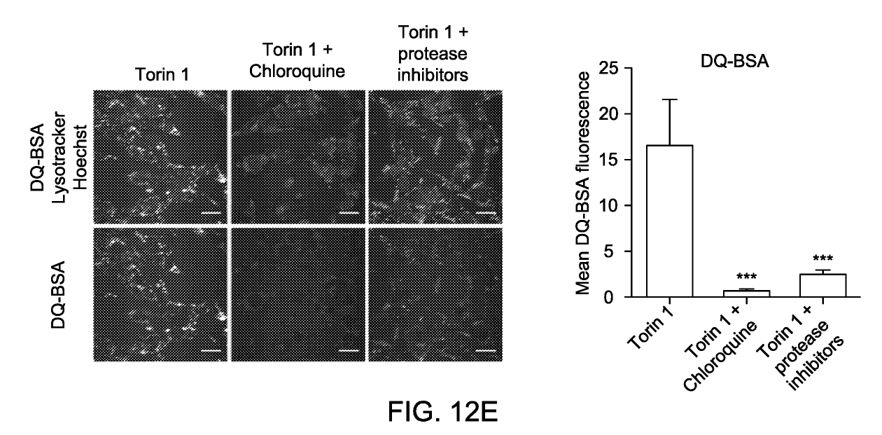

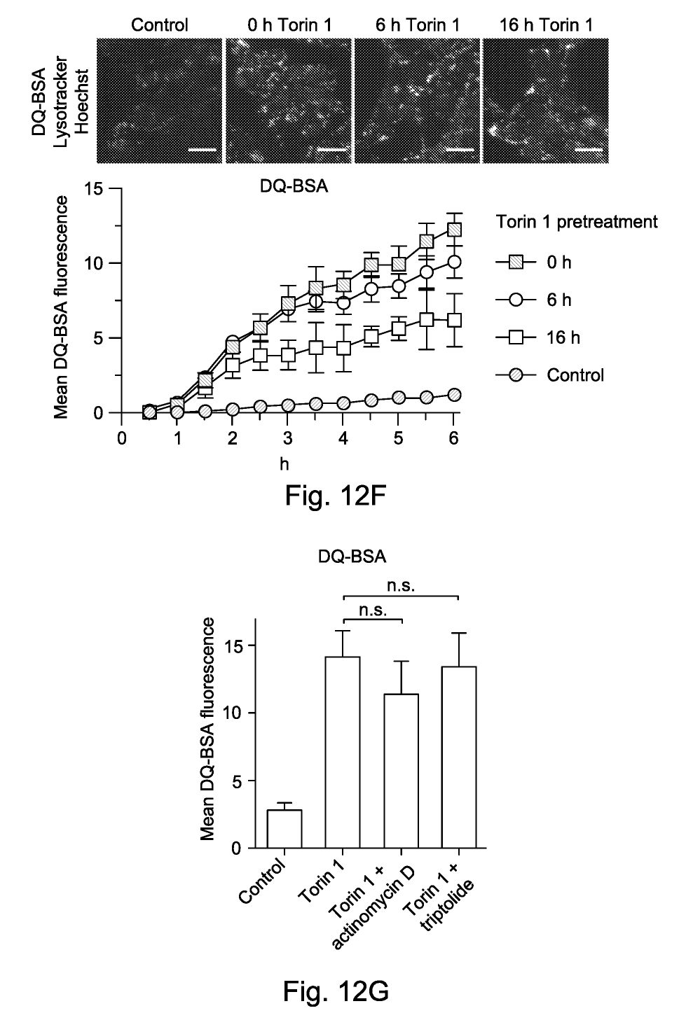

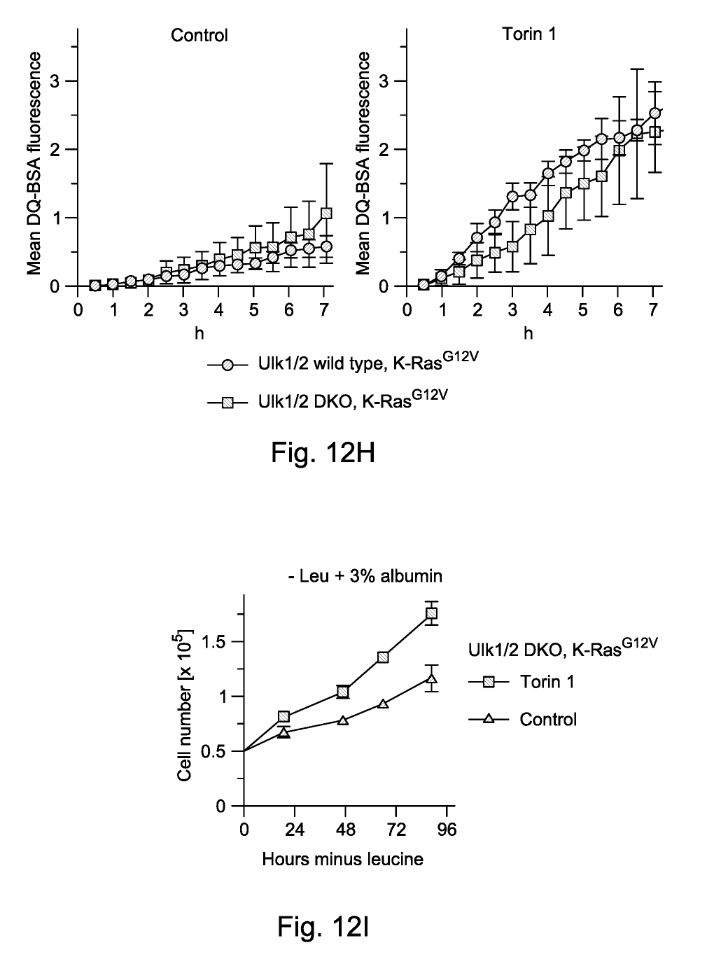

It was considered that the recovery of leucine from extracellular proteins constituted the rate-limiting step for cell growth in leucine-free medium. This suggested that mTORC1 suppresses extracellular protein-dependent growth by restricting a cell's access to the amino acid content of extracellular proteins, conceivably by blocking either their endocytosis or lysosomal degradation. To distinguish these possibilities, it was first tested whether mTORC1 inhibition increased internalization of extracellular macromolecules. However, neither torin 1 nor Raptor knockdown elevated cellular uptake of fluorescently labeled dextran or albumin, indicating that mTORC1 signaling does not inhibit bulk internalization of extracellular macromolecules (FIG. 12A-C). It was next examined whether mTORC1 inhibition affected lysosomal degradation of internalized proteins. K-Ras^{G12D} MEFs were placed in medium containing DQ-BSA and, to mark lysosomes, lysotracker±torin 1, and the fluorescent signal generated by lysosomal proteolysis of DQ-BSA was followed over time mTOR inhibition caused a dramatic increase in the fluorescence dequenching of DQ-BSA, indicative of its enhanced degradation (FIG. 11A, B). Similarly, inhibiting mTORC1 by Raptor knockdown increased cellular DQ-BSA fluorescence (FIG. 12D). Blocking lysosomal proteolysis with chloroquine or lysosomal protease inhibitors abrogated DQ-BSA degradation in torin 1-treated cells (FIG. 12E). Thus, mTORC1 negatively regulates lysosomal degradation of proteins that were taken up from the environment.

To further investigate how Ras and mTORC1 signaling regulated lysosomal proteolysis of internalized proteins, the effects of mTORC1 inhibition and K-Ras activation were compared. Inhibiting mTORC1 with torin 1 in wild type MEFs caused a significant increase in DQ-BSA fluorescence (FIG. 11C, D). Similarly, MEFs harboring the constitutively active K-Ras^{G12D} mutation displayed higher DQ-BSA degradation than wild type MEFs. Combining torin 1 treatment with constitutive K-Ras activation had synergistic effects and increased DQ-BSA proteolysis almost four times more than either manipulation alone. Thus, K-Ras enhances a cell's ability to take up and degrade extracellular proteins. In contrast, whereas mTORC1 specifically restricts their lysosomal degradation.

One mechanism by which mTORC1 signaling might impede degradation of internalized proteins is to suppress expression of genes involved in endocytosis or lysosomal biogenesis (Shimobayashi and Hall, 2014). To address whether mTORC1 inhibition enhanced DQ-BSA degradation through secondary effects of transcriptional changes, the time frame during which mTORC 1 inhibitors exerted their effect was determined. Pre-treating K-Ras^{G12D} MEFs with torin 1 for 6 h or 16 h did not increase the rate of DQ-BSA degradation (FIG. 12F). Moreover, when transcription was blocked by actinomycin D or triptolide, the torin 1-induced increase in DQ-BSA fluorescence was unaffected (FIG. 12G). Thus, lysosomal proteolysis of internalized proteins is an immediate cellular response to mTORC1 inhibition. Autophagic engulfment and degradation of intracellular constituents is suppressed by mTORC1 under nutrient-replete conditions by inhibition of the autophagy initiator kinases Ulk1/2 (He and Klionsky, 2009; Mizushima, 2010). As endocytosis and autophagy are both vesicular trafficking pathways that deliver macromolecules to the lysosome, the consequences of Ulk1/2 deletion on DQ-BSA proteolysis were examined. Ulk1/2 wild type and DKO MEFs expressing K-Ras^{G12V} degraded DQ-BSA at similar rates and responded to torin 1 treatment with comparable increase in DQ-BSA proteolysis (FIG. 12H). Moreover, torin 1 enhanced proliferation of Ulk1/2 DKO MEFs in leucine-free medium containing 3% albumin (FIG. 12I). Thus, mTORC1 suppresses lysosomal proteolysis of extracellular proteins by a mechanism that is distinct from its regulation of autophagy.

mTORC1 Signaling Can Have Opposite Effects on Cell Proliferation Depending on a Cell's Source of Amino Acids

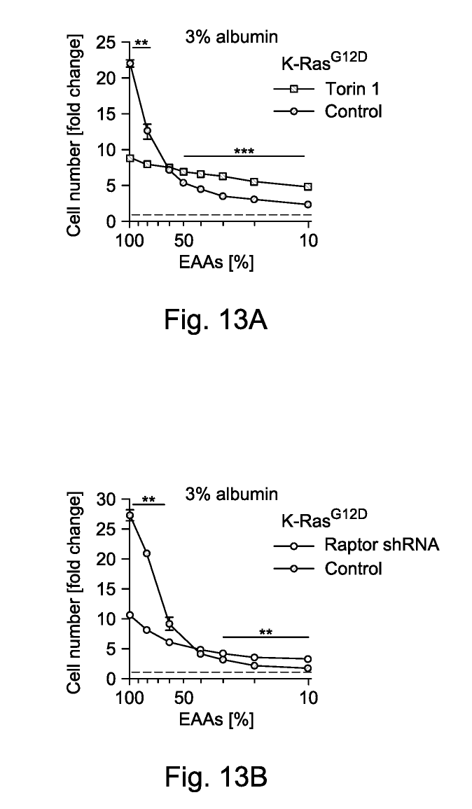

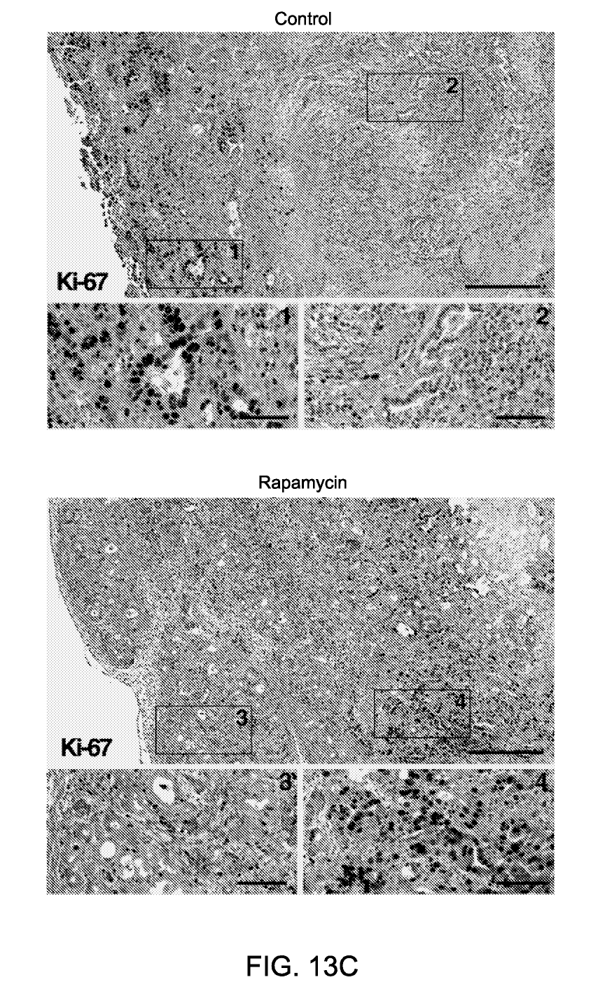

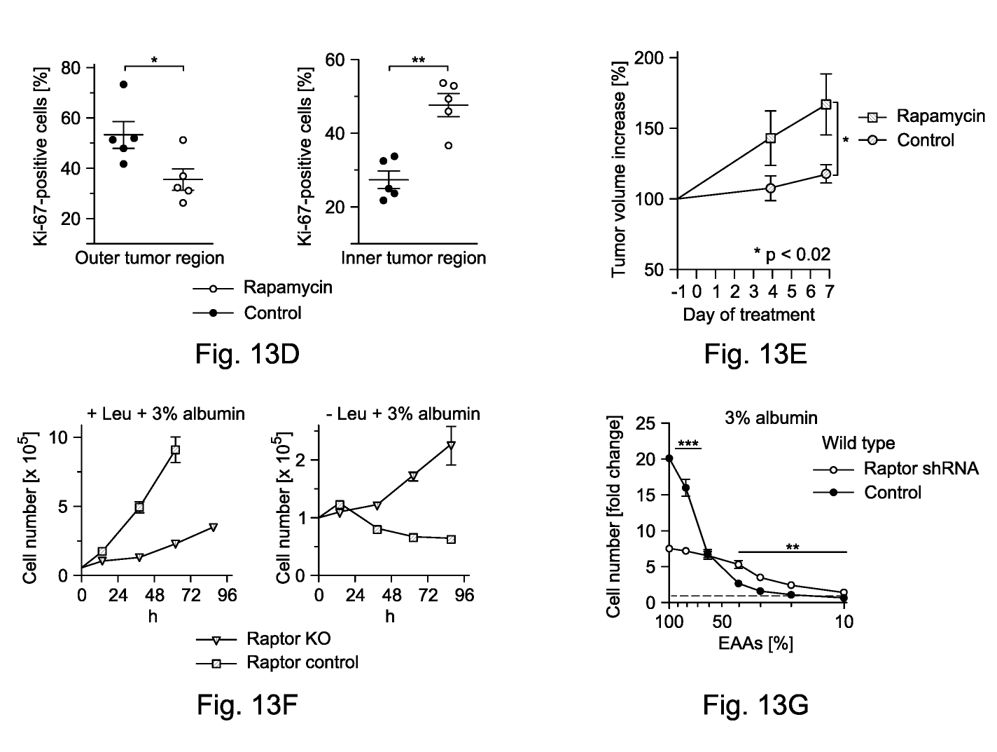

While it has been shown in many different systems that mTORC1 promotes growth when amino acids are abundant extracellularly, the above data indicated that mTORC1 could suppress cell growth that relies on the catabolism of extracellular proteins. This led to the investigation of the impact of mTORC1 inhibition on growth of K-Ras^{G12D} MEFs in medium containing decreasing amounts of EAAs, but supplemented with 3% albumin as an alternative EAA source. Torin 1 treatment or Raptor knockdown strongly decreased cell proliferation when EAAs were abundant extracellularly (FIG. 13A, B). However, whereas the proliferation of control cells quickly dropped with decreasing EAA levels, cells treated with torin 1 or expressing Raptor shRNA displayed only slightly reduced proliferation over a 10-fold range of EAA concentrations. At low EAA concentrations, torin 1 treatment or Raptor knockdown significantly improved cell proliferation. These results suggest that mTORC1 signaling stimulates proliferation of K-Ras^{G12D} MEFs in amino acid-rich medium, but restricts it under conditions when extracellular proteins become a required source of essential amino acids.

### Rapamycin Enhances the Growth of K-Ras-Induced Pancreatic Tumors In Vivo

To determine whether mTORC1 inhibition could promote proliferation of K Ras mutant cells in vivo, the effects of mTORC1 inhibition on the development of pancreatic ductal adenocarcinoma (PDA) were examined using a genetically engineered mouse model that expresses endogenous alleles of mutant K-Ras and p53 specifically in the pancreas (KPC) (Hingorani et al., 2005). In humans, K-Ras is mutated in the majority of pancreatic cancers; consistently, tumor cells of KPC mice were shown to internalize high molecular weight dextran through macropinocytosis, indicating that they have the potential to access extracellular proteins (Commisso et al., 2013). Moreover, the tumor microenvironment of pancreatic cancer is poorly vascularized and highly nutrient-depleted (Kamphorst et al. 2015), which raises the possibility that extracellular proteins could function as an important alternative nutrient source in this context.

KPC mice with established tumors of comparable size were treated over the course of 8 days with rapamycin, and cell proliferation was examined by Ki-67 staining of tumor tissue. Because mTORC1 inhibition enhanced cell growth in cultured cells specifically during amino acid starvation, the effects of rapamycin on proliferation of tumor cells in interior, hypovascularized tumor regions that were negative for the endothelial marker CD31 were examined. Indeed, rapamycin treatment caused a striking increase in the number of Ki-67-positive cells in those tumor regions, despite the absence of the mTORC1 downstream target, phosphorylated S6 (FIG. 13C, D; 14A). In contrast, rapamycin decreased the fraction of

Ki-67-positive cells in outer, vascularized tumor regions with a concomitant decrease in phospho-S6. These data indicate that pancreatic cancer cells respond to rapamycin in vivo depending on the tumor microenvironment they reside in—while rapamycin decreases cell proliferation in outer, vascularized regions, it enhances proliferation in interior, hypovascularized regions. These findings support the idea that mTORC1 can function as a suppressor of cell growth during nutrient starvation. Strikingly, rapamycin treatment significantly accelerated tumor growth in KPC mice (FIG. 13E).

Genetic Ablation of mTORC1 Signaling Can Induce Extracellular Protein-Dependent Growth Independently of Ras Transformation

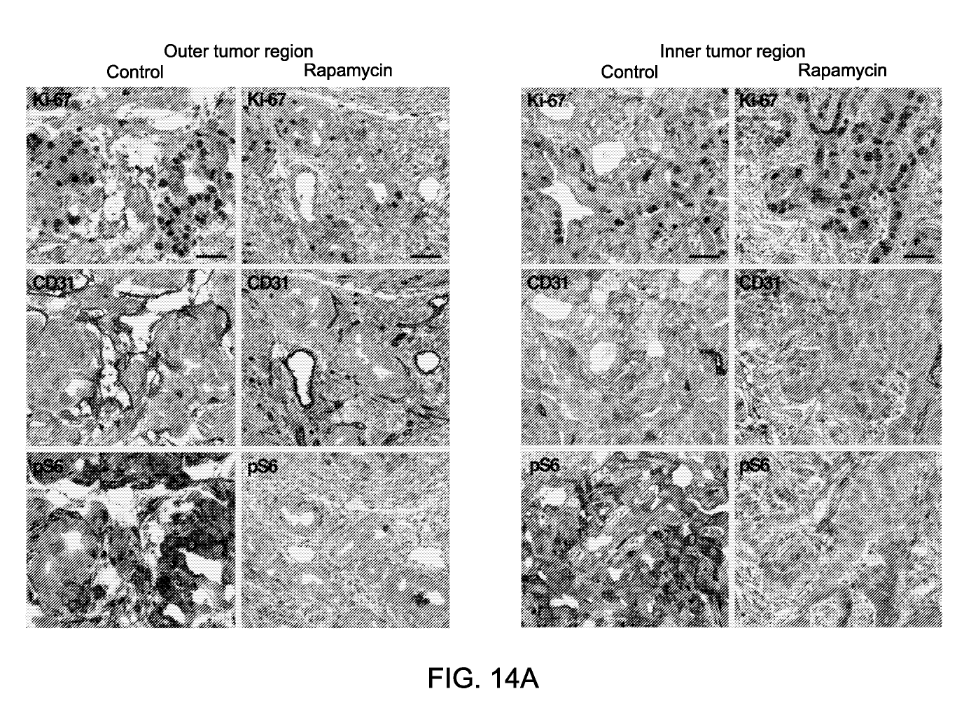

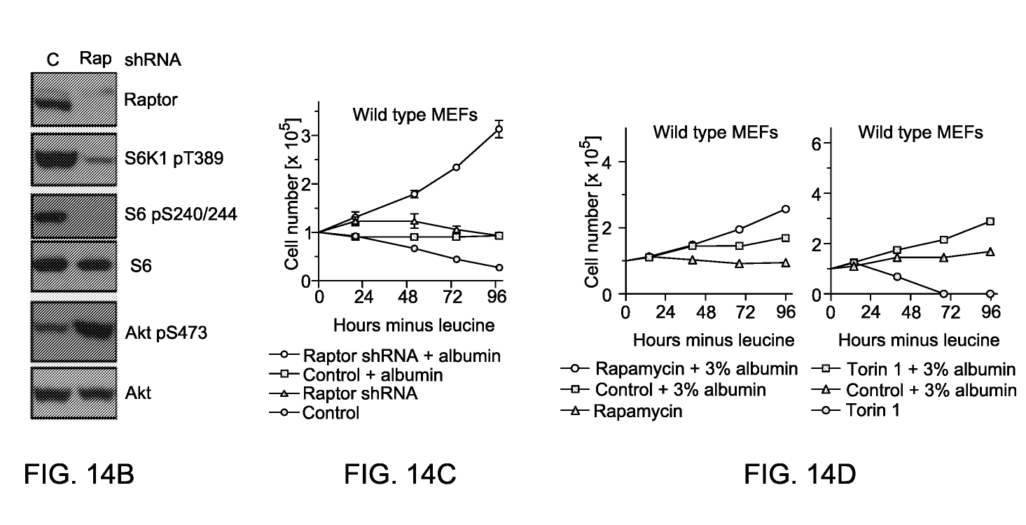

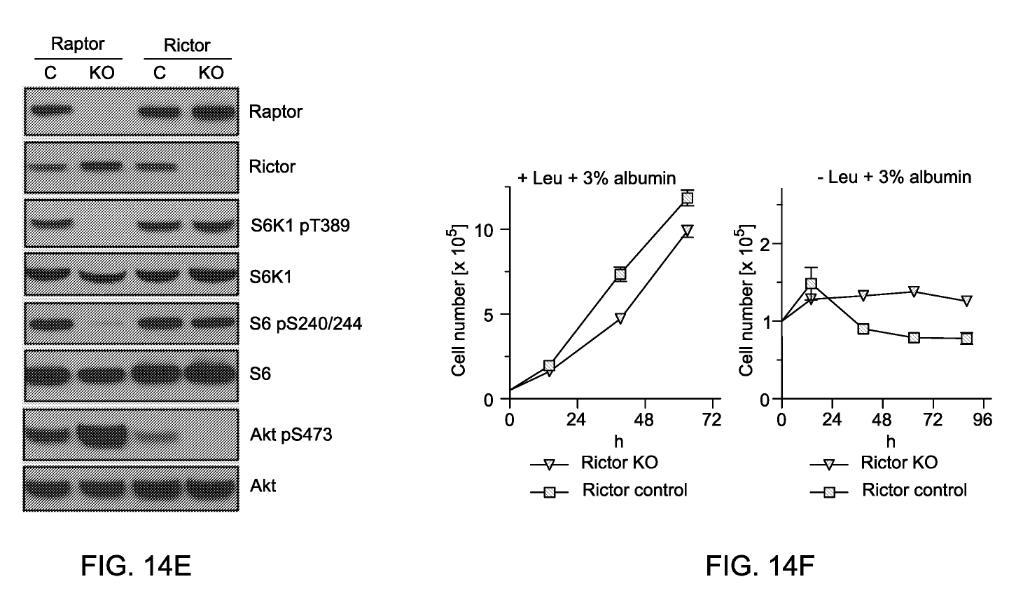

Finally, to determine if the role of mTORC1 in suppressing utilization of extracellular proteins as an amino acid source to support cell growth was restricted to Ras-transformed cells, the consequences of mTORC1 inhibition in cells harboring wild type Ras alleles were investigated. Wild type MEFs expressing Raptor shRNA or treated with mTOR inhibitors could robustly proliferate in leucine-free medium supplemented with 3% albumin, (FIG. 14B-D). To more stringently block mTOR signaling, Raptor or Rictor were genetically ablated from MEFs harboring conditional alleles (FIG. 14E) (Cybulski et al., 2012). While Raptor knockout cells displayed strongly decreased cell proliferation in nutrient-replete medium as compared to wild type controls, they could sustain proliferation in leucine-free medium +3% albumin (FIG. 13F). In contrast, deletion of Rictor only modestly decreased cell proliferation in leucine-containing medium and did not result in growth of leucine-deprived cells in albumin-supplemented medium (FIG. 14F). The proliferation of wild type MEFs expressing control or Raptor shRNA was also examined in medium containing decreasing amounts of EAAs as well as 3% albumin as an alternative EAA source. Raptor knockdown impaired cell proliferation under EAA-replete conditions (FIG. 13G). However, the difference in cell proliferation between control and Raptor knockdown cells diminished when EAA levels were reduced, and at low EAA levels, Raptor knockdown enhanced proliferation.

mTORC1 Suppresses the Utilization of Extracellular Proteins as Nutrients

The above results demonstrate that in mammalian cells mTORC1 signaling suppresses lysosomal catabolism of proteins that were taken up from the environment. As a corollary, mTORC1 inhibition enhances cell proliferation that relies on extracellular proteins as nutrients, for instance in cultured cells deprived of EAAs or pancreatic cancer cells residing in poorly vascularized tumor regions. It is known that the mTORC1 pathway is a potent stimulator of cell growth under nutrient-rich conditions, in part through enhancing translation (Ma and Blenis, 2009; Shimobayashi and Hall, 2014). However, the ability of mTORC1 to promote net protein synthesis strictly requires an exogenous source of amino acids. The present work indicates that by restricting amino acid recovery from extracellular proteins, mTORC1 couples cell growth to extracellular availability of free amino acids. This suggests that mTORC1 inhibition can promote growth under conditions when protein biosynthesis is limited by the acquisition of amino acids rather than the efficiency of translation. Whether mTORC1 stimulates or suppresses cell growth may therefore depend on a cell's amino acid source.

Previous work showed that inhibition of mTORC1 could support cell survival in the absence of a source of extracellular EAAs. When cells are deprived of leucine in the absence of extracellular proteins, the ensuing inactivation of mTORC1 leads to de-repression of the autophagy initiation kinases Ulk1/2, which trigger the formation of autophagosomes to engulf intracellular constituents for subsequent delivery to the lysosome (He and Klionsky, 2009; Mizushima, 2010). Through this mechanism, autophagy supports cell survival during leucine deprivation. However, catabolism of intracellular proteins cannot lead to net acquisition of leucine (or other EAAs) required for cell growth and proliferation. Rather, autophagic degradation of intracellular proteins recovers sufficient EAAs for cells to engage in adaptive protein synthesis to sustain cell survival during limited periods of nutrient deprivation. The work presented here demonstrates that mammalian cells can utilize extracellular proteins as a source of EAAs that allows sustained cell viability. If cells catabolize sufficient amounts of extracellular proteins, as a result of activating mutations in Ras and/or suppression of mTORC1 , they can even support net protein synthesis to increase in biomass and proliferate. The data presented here demonstrate that Ulk1/2 are dispensable for extracellular protein-dependent growth of Ras mutant cells. In fact, genetic deletion of Ulk1/2 enhances cell proliferation upon mTORC1 inhibition. This suggests that the degradation of intracellular constituents through autophagy as a result of mTORC1 inhibition can limit the rate at which amino acid-deprived cells can grow by taking up and degrading extracellular proteins.

Amino acids regulate mTORC1 activity by inducing its recruitment to lysosomal membranes (Sancak et al., 2010). The present work demonstrates that lysosomal degradation of endocytosed proteins regulates the mTORC1 pathway at the same step as amino acids that were imported from the environment in their monomeric form: both nutrients induce Rag-dependent recruitment of mTORC1 to lysosomal membranes, which permits its subsequent activation by growth factor signaling. Similarly, lysosomal catabolism of intracellular proteins that were delivered through autophagy leads to recruitment of mTORC1 to this organelle (Yu et al., 2010). Together, these data suggest that the regulation of mTORC1 activity at the lysosome constitutes a sensitive mechanism that allows cells to monitor amino acids recovered from proteins that were delivered through endocytosis or autophagy. Conversely, being activated at lysosomal membranes, mTORC1 may be well positioned to function as a regulator of cargo delivery to the lysosome by these membrane trafficking pathways. Notably, proteins that regulate endosomal trafficking have been recently emerging as a novel class of mTORC1 substrates (Hsu et al., 2011; Kim et al., 2015; Yu et al., 2011).

Implications for the Use of mTOR Inhibitors as Therapeutics

The present results may also shed light on the puzzling lack of efficacy of mTOR inhibitors as cancer therapeutics. There has been much effort over the last several years to target the mTORC1 pathway in cancer treatment. These studies have been motivated by the observation that human tumors often display elevated mTORC1 activity, commonly because of mutations in its upstream activator, the PI3-kinase pathway (Manning and Cantley, 2007). However, recent clinical trials showed only limited efficacy of rapamycin analogs (rapalogs) in a variety of solid tumors. The present work reveals an intrinsic weakness of mTOR inhibitors in cancer treatment. By enhancing catabolism of extracellularly derived proteins, mTOR inhibitors increase the use of extracellular proteins as alternative nutrients to support survival and sustain growth. This may be particularly important in nutrient-depleted tumor microenvironments or during metastasis, when tumor cells must adapt to novel metabolic niches. These findings predict that the growth promoting effects of mTOR inhibitors correlate with the capacity of cancer cells to take up sufficient extracellular proteins through endocytic pathways such as Ras-directed macropinocytosis. Consistently, treating KPC mice with rapamycin increases proliferation of pancreatic cancer cells that reside in poorly vascularized tumor regions and accelerates net tumor growth. This may explain the failure of mTOR inhibitors in the treatment of a variety of tumor types with activating mutations in Ras signaling, such as pancreatic cancer, in which K-Ras is the major driver oncogene, (Javle et al., 2010) or neurofibromatosis, which is caused by mutation in the Ras suppressor NF1.

One current explanation for the limited success of rapalogs is that they alleviate feedback repression of PI3-kinase/Akt signaling. This led to the development of active site mTOR inhibitors, which by targeting mTOR kinase inhibit mTORC1 and the Akt activator mTORC2, as well as dual mTOR/PI3-kinase inhibitors. The efficacy of these inhibitors in cancer therapy is currently being investigated, but the above data show that in cultured cells, the different classes of mTOR inhibitors share the caveat of promoting nutritional utilization of extracellular proteins. However, our work suggests that an alternative approach of combining mTOR inhibitors with inhibitors that block uptake or lysosomal degradation of extracellular proteins could achieve greater efficacy. When evaluating the role of mTORC1 signaling in tumorigenesis, it may also be worth considering insights from cancer genome sequencing efforts. Relatively few cancers were found to display mutations in mTOR kinase or direct pathway regulators that cause constitutive pathway activation. In contrast, mutations in the upstream PI3-kinase/Akt pathway are prevalent in human cancers (Manning and Cantley, 2007). This conceivably allows cancer cells to increase mTORC1 activity under nutrient-rich conditions, while not abrogating its regulation by amino acids, thus allowing tumor cells to adapt to nutrient deprivation.

Macropinocytosis of Extracellular Macromolecules as a Strategy of Nutrient Acquisition inEukaryotic Cells

The present work demonstrates that mammalian cells can tap extracellular proteins as a source of EAAs to sustain survival and proliferation. These data support and extend recent work showing that Ras directed protein macropinocytosis could alleviate the dependence of transformed cells on extracellular glutamine supply (Commisso et al., 2013). Ras-induced macropinocytosis of extracellular proteins has been previously recognized to sustain growth of unicellular amoeboid eukaryotes such as Dictyostelium discoideum (Amyere et al., 2002). Taken together, these findings indicate that micropinocytosis constitutes an evolutionarily ancient strategy of nutrient acquisition in eukaryotic cells. Interestingly, macropinocytosis in metazoan cells is under control of growth factor signaling, and micropinocytosis induction is an immediate response of various mammalian cell types to serum stimulation (Amyere et al., 2002; Mercer and Helenius, 2009). This suggests that growth factors can instruct cells to take up not only low molecular weight nutrients such as glucose and amino acids, but also extracellular macromolecules. However, concerted inputs from growth factor signaling and amino acids result in mTORC1 activation, which limits catabolism of proteins internalized from the environment. Therefore, mTORC1 initiates anabolic cellular metabolism that utilizes free amino acids, while preventing degradation of extracellularly derived proteins. This mechanism conceivably prevents futile turnover of the proteins contained in body fluids, as long as monomeric amino acids are available.

### REFERENCES

Amyere, M., Mettlen, M., Van Der Smissen, P., Platek, A., Payrastre, B., Veithen, A., and Courtoy, P. J. (2002). Origin, originality, functions, subversions and molecular signalling of macropinocytosis. International journal of medical microbiology: IJMM 291, 487-494. Bar-Sagi, D., and Feramisco, J. R. (1986). Induction of membrane ruffling and fluid-phase pinocytosis in quiescent fibroblasts by ras proteins. Science (New York, N.Y.) 233, 1061-1068.
Bligh, E. G., and Dyer, W. J. (1959). A rapid method of total lipid extraction and purification. Canadian journal of biochemistry and physiology 37, 911-917.
Cairns, R. A., Harris, I. S., and Mak, T. W. (2011). Regulation of cancer cell metabolism. Nature reviews Cancer 11, 85-95.
Cheong, H., Lindsten, T., Wu, J., Lu, C., and Thompson, C. B. (2011). Ammonia-induced autophagy is independent of ULK1/ULK2 kinases. Proceedings of the National Academy of Sciences of the United States of America 108, 11121-11126.
Commisso, C., Davidson, S. M., Soydaner-Azeloglu, R. G., Parker, S. J., Kamphorst, J. J., Hackett, S., Grabocka, E., Nofal, M., Drebin, J. A., Thompson, C. B., et al. (2013). Macropinocytosis of protein is an amino acid supply route in Ras-transformed cells. Nature 497, 633-637.
Cybulski, N., Zinzalla, V., and Hall, M. N. (2012). Inducible raptor and rictor knockout mouse embryonic fibroblasts. Methods in molecular biology (Clifton, N.J.) 821, 267-278. Edinger, A. L., and Thompson, C. B. (2002). Akt maintains cell size and survival by increasing mTOR dependent nutrient uptake. Molecular biology of the cell 13, 2276-2288.
Fellmann, C., Zuber, J., McJunkin, K., Chang, K., Malone, C. D., Dickins, R. A., Xu, Q., Hengartner, M. O., Elledge, S. J., Hannon, G. J., et al. (2011). Functional identification of optimized RNAi triggers using a massively parallel sensor assay. Molecular cell 41, 733-746.
He, C., and Klionsky, D. J. (2009). Regulation mechanisms and signaling pathways of autophagy. Annual review of genetics 43, 67-93.
Hingorani, S. R., Wang, L., Multani, A. S., Combs, C., Deramaudt, T. B., Hruban, R. H., Rustgi, A. K., Chang, S., and Tuveson, D. A. (2005). Trp53R172H and KrasG12D cooperate to promote chromosomal instability and widely metastatic pancreatic ductal adenocarcinoma in mice. Cancer cell 7, 469-483.
Hsu, P. P., Kang, S. A., Rameseder, J., Zhang, Y., Ottina, K. A., Lim, D., Peterson, T. R., Choi, Y., Gray, N. S., Yaffe, M. B., et al. (2011). The mTOR-regulated phosphoproteome reveals a mechanism of mTORC1 -mediated inhibition of growth factor signaling. Science (New York, N.Y.) 332, 1317-1322.
Javle, M. M., Shroff, R. T., Xiong, H., Varadhachary, G. A., Fogelman, D., Reddy, S. A., Davis, D., Zhang, Y., Wolff, R. A., and Abbruzzese, J. L. (2010). Inhibition of the mammalian target of rapamycin (mTOR) in advanced pancreatic cancer: results of two phase II studies. BMC cancer 10, 368.
Jewell, J. L., Kim, Y. C., Russell, R. C., Yu, F. X., Park, H. W., Plouffe, S. W., Tagliabracci, V. S., and Guan, K. L. (2015). Metabolism. Differential regulation of mTORC1 by leucine and glutamine. Science (New York, N.Y.) 347, 194-198.
Kamphorst, J. J., Nofal, M., Commisso, C., Hackett, S. R., Lu, W., Grabocka, E., Vander Heiden, M. G., Miller, G., Drebin, J. A., Bar-Sagi, D., et al. (2015). Human pancreatic cancer tumors are nutrient poor and tumor cells actively scavenge extracellular protein. Cancer research 75, 544-553.
Kang, S. A., Pacold, M. E., Cervantes, C. L., Lim, D., Lou, H. J., Ottina, K., Gray, N. S., Turk, B. E., Yaffe, M. B., and Sabatini, D. M. (2013). mTORC1 phosphorylation sites encode their sensitivity to starvation and rapamycin. Science (New York, N.Y.) 341, 1236566.
Kim, E., Goraksha-Hicks, P., Li, L., Neufeld, T. P., and Guan, K. L. (2008). Regulation of TORC1 by Rag GTPases in nutrient response. Nature cell biology 10, 935-945.
Kim, Y. M., Jung, C. H., Seo, M., Kim, E. K., Park, J. M., Bae, S. S., and Kim, D. H. (2015). mTORC1 phosphorylates UVRAG to negatively regulate autophagosome and endosome maturation. Molecular cell 57, 207-218.
Lito, P., Saborowski, A., Yue, J., Solomon, M., Joseph, E., Gadal, S., Saborowski, M., Kastenhuber, E., Fellmann, C., Ohara, K., et al. (2014). Disruption of CRAF-mediated MEK activation is required for effective MEK inhibition in KRAS mutant tumors. Cancer cell 25, 697-710.
Ma, X. M., and Blenis, J. (2009). Molecular mechanisms of mTOR-mediated translational control. Nature reviews Molecular cell biology 10, 307-318.
Manning, B. D., and Cantley, L. C. (2007). AKT/PKB signaling: navigating downstream. Cell 129, 1261-1274.
Mercer, J., and Helenius, A. (2009). Virus entry by macropinocytosis. Nature cell biology 11, 510-520.
Mizushima, N. (2010). The role of the Atg1/ULK1 complex in autophagy regulation. Current opinion in cell biology 22, 132-139.
Nicklin, P., Bergman, P., Zhang, B., Triantafellow, E., Wang, H., Nyfeler, B., Yang, H., Hild, M., Kung, C., Wilson, C., et al. (2009). Bidirectional transport of amino acids regulates mTOR and autophagy. Cell 136, 521-534.
Pylayeva-Gupta, Y., Grabocka, E., and Bar-Sagi, D. (2011). RAS oncogenes: weaving a tumorigenic web. Nature reviews Cancer 11, 761-774.
Reis, R. C., Sorgine, M. H., and Coelho-Sampaio, T. (1998). A novel methodology for the investigation of intracellular proteolytic processing in intact cells. European journal of cell biology 75, 192-197.
Sancak, Y., Bar-Peled, L., Zoncu, R., Markhard, A. L., Nada, S., and Sabatini, D. M. (2010). Ragulator-Rag complex targets mTORC1 to the lysosomal surface and is necessary for its activation by amino acids. Cell 141, 290-303.
Sancak, Y., Peterson, T. R., Shaul, Y. D., Lindquist, R. A., Thoreen, C. C., Bar-Peled, L., and Sabatini, D. M. (2008). The Rag GTPases bind raptor and mediate amino acid signaling to mTORC1. Science (New York, N.Y.) 320, 1496-1501.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nature methods 9, 676-682.
Shimobayashi, M., and Hall, M. N. (2014). Making new contacts: the mTOR network in metabolism and signalling crosstalk. Nature reviews Molecular cell biology 15, 155-162.
Thompson, C. B. (2011). Rethinking the regulation of cellular metabolism. Cold Spring Harbor symposia on quantitative biology 76, 23-29.
Thoreen, C. C., Kang, S. A., Chang, J. W., Liu, Q., Zhang, J., Gao, Y., Reichling, L. J., Sim, T., Sabatini, D. M., and Gray, N. S. (2009). An ATP-competitive mammalian target of rapamycin inhibitor reveals rapamycin resistant functions of mTORC1. The Journal of biological chemistry 284, 8023-8032.
Tuveson, D. A., Shaw, A. T., Willis, N. A., Silver, D. P., Jackson, E. L., Chang, S., Mercer, K. L., Grochow, R., Hock, H., Crowley, D., et al. (2004). Endogenous oncogenic K-ras(G12D) stimulates proliferation and widespread neoplastic and developmental defects. Cancer cell 5, 375-387.
West, M. A., Bretscher, M. S., and Watts, C. (1989). Distinct endocytotic pathways in epidermal growth factor-stimulated human carcinoma A431 cells. The Journal of cell biology 109, 2731-2739.
Yu, L., McPhee, C. K., Zheng, L., Mardones, G. A., Rong, Y., Peng, J., Mi, N., Zhao, Y., Liu, Z., Wan, F., et al . (2010). Termination of autophagy and reformation of lysosomes regulated by mTOR. Nature 465, 942-946.
Yu, Y., Yoon, S. O., Poulogiannis, G., Yang, Q., Ma, X.M., Villen, J., Kubica, N., Hoffman, G. R., Cantley, L. C., Gygi, S. P., et al. (2011). Phosphoproteomic analysis identifies Grb10 as an mTORC1 substrate that negatively regulates insulin signaling. Science (New York, N.Y.) 332, 1322-1326.
Yun, J., Rago, C., Cheong, I., Pagliarini, R., Angenendt, P., Rajagopalan, H., Schmidt, K., Willson, J. K., Markowitz, S., Zhou, S., et al. (2009). Glucose deprivation contributes to the development of KRAS pathway mutations in tumor cells. Science (New York, N.Y.) 325, 1555-1559.
Zuber, J., McJunkin, K., Fellmann, C., Dow, L. E., Taylor, M. J., Hannon, G. J., and Lowe, S. W. (2011). Toolkit for evaluating genes required for proliferation and survival using tetracycline-regulated RNAi. Nature biotechnology 29, 79-83.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims

We claim:

1. A method comprising steps of:
administering to a subject suffering from a cancer characterized by oncogenic activation of Ras protein a therapeutic regimen comprising
(i) an mTORC inhibition therapy and
(ii) a toxin therapy.

2. The method of claim 1, wherein mTORC inhibition therapy is administered prior to the toxin therapy.

3. The method of claim 1, wherein the mTORC inhibition therapy comprises an mTORC1 inhibitor.

4. The method of claim 1, wherein the cancer comprises a solid tumor.

5. The method of any of claims 1 to 4, wherein the cancer resides in a microenvironment that is desmoplastic and/or hypovascularized.

6. The method of any of claims 1 to 5, wherein the cancer comprises metastatic cells.

7. The method of any of claims 1 to 6, wherein the cancer is pancreatic cancer.

8. The method of any of claims 1 to 7, wherein the Ras protein is selected from the group consisting of K-Ras, H-Ras, N-Ras, and combinations thereof.

9. The method of any of claims 2 to 8, wherein the mTORC1 inhibitor is selected from the group consisting of rapamycin/sirolimus, everolimus, temsirolimus, umirolimus, zotarolimus, deforolimus, wortmannin, TOP-216, TAFA93, CCI-779, ABT578, SAR543, ascomycin, FK506, AP23573, AP23464, AP23841, KU-0063794, INK-128, EX2044, EX3855, EX7518, AZD-8055, AZD-2014, Palomid 529, Pp-242, OSI-027 and combinations thereof

10. The method of any of claims 1 to 9, wherein the toxin therapy is selected from the group consisting of cyclophosphamide, chlorambucil, cisplatin, busulfan, melphalan, carmustine, streptozotocin, triethylenemelamine, mitomycin C, methotrexate, etoposide, 6-mercaptopurine, 6-thiocguanine, cytarabine, 5-fluorouracil, dacarbazine, actinomycin D, doxorubicin, daunorubicin, bleomycin, mithramycin, vincristine, vinblastine, paclitaxel, pactitaxel derivatives, cytostatic agents, dexamethasone, prednisone, hydroxyurea, asparaginase, leucovorin, amifostine, dactinomycin, mechlorethamine, streptozocin, cyclophosphamide, lomustine, doxorubicin lipo, gemcitabine, daunorubicin lipo, procarbazine, mitomycin, docetaxel, aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil, and combinations thereof

11. The method of claim 10 wherein the toxin therapy is administered at a low dose.

12. The method of any of claims 1 to 10, wherein the toxin is conjugated to a soluble protein.

13. The method of claim 12, wherein the soluble protein is albumin.

14. The method of any of claims 1 to 13, wherein the therapeutic regimen does not comprise a lysosomal inhibition therapy.

15. The method of any of claims 1 to 14, wherein the therapeutic regimen does not comprise a Ras inhibition therapy.

16. A method for identifying a cancer that is likely to respond favorably to treatment with an mTORC1 inhibitor as a monotherapy, the method comprising a step of assaying a sample from the cancer for oncogenic activation of Ras, wherein the sample is determined to have low or no oncogenic Ras activity.

17. A method for identifying a cancer that is likely to not respond favorably to treatment with mTORC1 inhibitor as a monotherapy, the method comprising a step of assaying a sample from the cancer for oncogenic activation of Ras, wherein the sample is determined to have oncogenic Ras activity.

18. The method of claim 17, wherein oncogenic activation of Ras comprises constitutively active Ras caused by a genetic mutation.

19. The method of claim 18, wherein the genetic mutation comprises a mutated Ras protein.

20. The method of claim 18, wherein the genetic mutation results in decreased expression or activity of a Ras suppressor protein.

21. The method of any of claims 17 to 20, wherein oncogenic activation of Ras is detected by allele-specific polymerase chain reaction (PCR), PCR and Sanger dideoxy sequencing, PCR and pyrosequencing, PCR and mass spectrometry (MS), PCR and single base extension, multiplex ligation-dependent probe amplification (MLPA), or fluorescence in situ hybridization (FISH).

22. A composition for detection of cancer, comprising an imaging agent conjugated to a substrate for macropinocytosis by a cancer cell.

23. The composition of claim 22, further comprising an mTORC1 inhibitor.

24. The composition of any of claims 22 to 23, wherein the cancer is detected in vivo in a subject.

25. The composition of any of claims 22 to 24, wherein the imaging agent is metallic.

26. The composition of any of claims 22 to 24, wherein the imaging agent is radiolabeled.

27. The composition of any of claims 22 to 26, wherein the substrate for macropinocytosis comprises a soluble protein.

28. The composition of claim 27, wherein the soluble protein is albumin.

29. The composition of claim 22, wherein the cancer exhibits oncogenic Ras activity.

30. A method for detecting cancer in a subject, the method comprising steps of:
(i) administering to the subject an mTORC1 inhibitor;
(ii) administering to the subject an imaging agent capable of eliciting a detectable signal; and
(iii) detecting the signal in elicited by the imaging agent.

31. The method of claim 30, wherein the imaging agent is conjugated to a substrate for macropinocytosis by a cancer cell.

32. The method of claim 31, wherein the imaging agent is conjugated to a soluble protein.

33. The method of claim 32, wherein the soluble protein is albumin.

34. The method of any of claims 30 to 33, wherein the mTORC1 inhibitor is administered prior to the imaging agent.

35. The method of claim 30 wherein the mTORC1 inhibitor is administered to the subject at least 1, 3, 5, 12, or 24 hours prior to the imaging agent.
